(19) United States
(12) Patent Application Publication
(10) Pub. No.: US 2009/0005713 A1
(43) Pub. Date: Jan. 1, 2009

(54) Method and Device for Using Vibroacoustical Stimulation to Treat Target Tissue Areas of Living Organisms

(76) Inventors: Yury M. Podrazhansky, Johns Creek, GA (US); Mikhail N. Lyubich, Johns Creek, GA (US)

Correspondence Address: MEHRMAN LAW OFFICE, P.C., P.O. Box 420797, ATLANTA, GA, 30342, US

(21) Appl. No.: 12/209,412

(22) Filed: Sep. 12, 2008

Related U.S. Application Data
(63) Continuation-in-part of application No. 12/029,885, filed on Feb. 12, 2008.
(60) Provisional application No. 60/889,355, filed on Feb. 12, 2007.

(51) Int. Cl. A61H 1/00; A61B 19/00

(52) U.S. Cl. 601/2; 128/898

(57) ABSTRACT

A method and device for using topically applied acoustical vibrations to treat diseases and conditions in living organisms. This approach is non-invasive, and more specifically does not involve introducing chemicals or physically invading the organisms. One or more acoustical transducers are placed directly on the skin of the organism in certain locations, and specific vibration profiles designed to treat specific diseases and conditions are applied to the organism through the transducers. The treatment includes the regular application of various vibration pulse profiles that generally include sequences of pulses in which each pulse has a duration in the range of 0.5 to 10 seconds, is separated by rest periods in the range of 0.1 to 3 seconds, is modulated with an oscillatory signal in the frequency range of 1 Hz to 1,500 Hz, and has a pulse amplitude in the range of range from about 20 to 5000 microns.

### REFERENCE TO RELATED APPLICATIONS

This application claims priority to U.S. Provisional Patent Application Ser. No. 60/889,355 entitled “Method for Increasing Production of Adult Stem Cells In Vivo” filed Feb. 12, 2007, and U.S. patent application Ser. No. 12/029,885 entitled “Method and Device for Using Vibroacoustic Stimulation to Enhance the Production of Adult Stem Cells in Living Organisms” filed Feb. 12, 2008, which are incorporated herein by reference.

### FIELD OF INVENTION

This invention relates to methodologies for treating diseases and conditions in living organisms without chemical or physical invasion into the organism and, more particularly, to a method and device for using acoustic vibrations to treat diseases and conditions in living organisms.

### BACKGROUND OF THE INVENTION

An adult stem cell is an undifferentiated cell found among differentiated cells in a tissue or organ that is capable of renewing itself and differentiating to yield the major specialized cell types of the tissue or organ. The primary roles of adult stem cells in a living organism are to maintain and repair the tissue in which they are found. Scientists have found adult stem cells in many more tissues than they once thought possible. This finding has led scientists to ask whether adult stem cells could be used for transplants.

Hematopoietic cell transplantation is the gold standard for cell-based therapy and is routinely used to treat a wide variety of blood disorders and cancer. A major limitation exists, however, in finding donors whose immune systems are compatible with those of the patients requiring transplantation. Therefore, there is a continuing need for techniques for stimulating the production of adult stem cells in living organisms.

Certain kinds of adult stem cells seem to have the multipotent hematopoietic ability to differentiate into a number of different cell types, given the right conditions. If this differentiation of adult stem cells can be controlled in the laboratory, these cells may become the basis of therapies for many serious common diseases. Scientists in many laboratories are trying to find ways to grow adult stem cells in cell cultures and manipulate them to generate specific cell types so they can be used to treat injury or disease. Some examples of potential treatments include replacing the dopamine-producing cells in the brains of Parkinson's patients, developing insulin-producing cells for type I diabetes, and repairing damaged heart muscle following a heart attack with cardiac muscle cells.

One population, known as hematopoietic stem cells, forms all the types of blood cells in the body. A second population, known as bone marrow stromal cells, was discovered a few years later. Stromal cells are a mixed cell population that generate bone, cartilage, fat, and fibrous connective tissue.

One important point to understand about adult stem cells is that there are a very small number of stem cells in each tissue. Stem cells are thought to reside in a specific area of each tissue where they may remain quiescent (non-dividing) for many years until they are activated by disease or tissue injury. The adult tissues reported to contain stem cells include the brain, bone marrow, peripheral blood, blood vessels, skeletal muscle, skin and liver. In particular, bone marrow stromal cells (mesenchymal stem cells) give rise to a variety of cell types: bone cells (osteocytes), cartilage cells (chondrocytes), fat cells (adipocytes), and other kinds of connective tissue cells such as those in tendons.

Stem cells differ from other kinds of cells in the body. All stem cells, regardless of their source, have three general properties: they are capable of dividing and renewing themselves for long periods; they are unspecialized; and they can give rise to specialized cell types. Stem cells have two important characteristics that distinguish them from other types of cells. First, they are unspecialized cells that renew themselves for long periods through cell division. The second is that under certain physiologic or experimental conditions, they can be induced to differentiate into cells with special functions, such as the beating cells of the heart muscle or the insulin-producing cells of the pancreas. Until now, the differentiation of adult stem cells controlled in the laboratory has been the only technique for developing adult stem cells for therapeutic uses for many serious common diseases.

Many serious diseases and disorders, and some therapies, involve damage to body tissues and/or insufficient natural repair of damaged body tissues. For example, cancer chemotherapy and radiation therapy destroy many other non-cancerous cells in the body, including those of the immune system. Disorders or cancers of the blood often involve abnormal growth and/or destruction of certain types of blood cells. Heart failure, which is currently incurable, often involves damage to heart muscle, which the body cannot repair. Liver failure often involves progressive destruction of liver cells. Stroke often involves irreversible damage and/or death of brain cells resulting from a lack of oxygen and nutrient-carrying blood to the affected portion of the brain. Type 2 diabetes, the most common form of the endocrine disorder, involves a progressive decrease in the ability of the pancreas to produce insulin, and its complications are due to progressive destruction of tissues in the eye (diabetic retinopathy, which can lead to blindness), kidney (diabetic nephropathy, which can lead to kidney failure), and nerves (diabetic neuropathy, which can lead to decreased sensation in the limbs and limb amputation as well as dysfunction of stomach, bladder). Osteoarthritis involves destruction of cartilage tissue in the joints. Parkinson's disease, Alzheimer's disease and other central nervous system disorders involve destruction of certain neurons in the brain. Various autoimmune disorders involve immune system attack and destruction of the lining around nerves (multiple sclerosis), the cell lining of the intestine (ulcerative colitis), cartilage in joints (rheumatoid arthritis), and other specific tissues for specific diseases. Spinal cord injuries involve trauma and destruction of nerve tissue in the spinal cord. Aging itself involves a general deterioration throughout the body's tissues.

Importantly, stem cell therapy offers the potential to help repair and renew the damaged tissues associated with these and other diseases, disorders and therapies. At present, it has been established that adult stem cells typically generate the cell types of tissue in which they reside. A blood-forming adult stem cell in the bone marrow, for example, normally gives rise to the many types of blood cells such as red blood cells, white blood cells and platelets. Until recently, it had been thought that a blood-forming cell in the bone marrow—which is called a hematopoietic stem cell—could not give rise to the cells of a very different tissue, such as nerve cells in the brain. However, a number of experiments over the last several years have raised the possibility that stem cells from one tissue may be able to give rise to cell types of a completely different tissue, a phenomenon known as “stem cell plasticity.” Examples of stem cell plasticity include blood stem cells differentiating to become neurons, liver stem cells differentiating to produce insulin, and hematopoietic stem cells differentiating to become heart muscle. Therefore, exploring the possibility of using adult stem cells for cell-based therapies has become a very active area of investigation by researchers.

Csete, et al., U.S. Pat. No. 6,759,242 issued Jul. 6, 2004 relates to the growth of cells in culture under conditions that promote cell survival, proliferation, and/or cellular differentiation. This patent contends that proliferation was promoted and apoptosis reduced when cells were grown in lowered oxygen as compared to environmental oxygen conditions traditionally employed in cell culture techniques.

Csete, et al., U.S. Pat. No. 6,610,540 issued Aug. 26, 2003 relates to the growth of cells in culture under conditions that promote cell survival, proliferation, and/or cellular differentiation. Again, this patent contends that proliferation was promoted and apoptosis reduced when cells were grown in lowered oxygen as compared to environmental oxygen conditions traditionally employed in cell culture techniques.

Csete, et al., U.S. Pat. No. 6,589,728 issued Jul. 8, 2003 describes a method of isolating, maintaining, and/or enriching stem or progenitor cells derived from diverse organ or tissue sources. This patent specifically teaches that these objectives can be accomplished by the controlled use of subatmospheric oxygen culture, and that the precise oxygen level or levels must be determined empirically and/or by reference to physiologic levels within intact functioning organ or tissue.

Shutko et al., Russian Patent No. 2,166,924 issued May 20, 2001 describes the application of micro-vibration treatment to eight to ten points located on central line of the vertebral column to mobilize existing adult stem cells in the blood, and thereby increase the presence adult stem cells in peripheral circulation. The micro-vibration frequencies applied to these areas is smoothly y changed within a particular acoustic bandwidth, the treatment duration is ten to fifteen minutes, and the increase in the presence of the adult stem cells in the peripheral circulation is expected to occur within three to four hours after application.

Gillis., U.S. Pat. No. 5,199,942 issued Apr. 6, 1993 relates generally to methods for autologous hematopoietic cell transplantation in patients undergoing cytoreductive therapies, and particularly to methods in which bone marrow or peripheral blood progenitor cells are removed from a patient prior to myelosuppressive cytoreductive therapy, expanded in ex-vivo culture in the presence of a growth factor, and then readministered to the patient concurrent with or following cytoreductive therapy to counteract the myelosuppressive effects of such therapy. The patent also describes a culture media containing one or more growth factors for expanding progenitor cells in ex-vivo culture.

Emerson, et al., U.S. Pat. No. 5,646,043 issued Jul. 8, 1997 describes methods, including culture media conditions, which provide for ex-vivo human stem cell division and/or the optimization of human hematopoietic progenitor cell cultures and/or increasing the metabolism or GM-CSF secretion or IL-6 secretion of human stromal cells are disclosed.

Bachovchin, et al., U.S. Pat. No. 6,258,597 issued Jul. 10, 2001 describes methods, compositions, and devices for chemically stimulating the number and/or differentiation of hematopoietic cells in living organisms. The methods involve contacting the hematopoietic cells with an inhibitor of dipeptidyl peptidase (DPIV) in the absence of exogenously provided cytokines.

Buck, et al., U.S. Pat. No. 7,037,719 issued May 2, 2006 describes enriched neural stem and progenitor cell populations, and methods for identifying, isolating and enriching for neural stem cells using reagent that bind to cell surface markers.

Saito, et al., U.S. Pat. No. 7,037,892 issued May 2, 2006 describes a method for chemically stimulating the proliferation a hematopoietic stem cells in a living organism. More particularly, the invention relates to a hematopoietic stem cell proliferating agent comprising insulin-like growth factor, either alone or in combination with some or other colony-stimulating factors and/or growth factors and to a method for proliferating.

Yang, U.S. Pat. No. 7,048,922 issued May 23, 2006 describes the stimulation of hematopoiesis by ex-vivo activated immune cells including a protocol for activating and administering human blood cells so that bone marrow histology and/or blood cell counts of patients suffering from aplastic anemia approach normal. The protocol includes culturing the blood cells in the presence of a cytokine and an ionophore.

Wallner, et al., U.S. Pat. No. 7,067,489 issued Jun. 27, 2006 describes methods and products for stimulating hematopoiesis, preventing low levels of hematopoietic cells and producing increased numbers of hematopoietic and mature blood cells both in-vivo and in-vitro.

Although these references indicate a high level of interest in in-vivo and in-vitro techniques for stimulating the production of stem cells, only Shutko et al., Russian Patent No. 2,166,924, describes the topical use of acoustical vibrations for stimulating the production of adult stem cells. However, the techniques described in this application are directed to mobilizing existing adult stem cells in the blood to increase the presence adult stem cell in peripheral circulation. The effect of the acoustical vibration treatment is expected to occur within about three to four hours after application. Therefore, Shutko et al. is directed to mobilizing existing adult stem cells, and does not describe a technique for stimulating the production of new adult stem cells in a living organism.

In view of the foregoing, it will be appreciated none of the conventional technologies provide a non-invasive technique for stimulating the production of adult stem cells in living organisms. Accordingly, there remains a need in the art for techniques for stimulating the production of adult stem cells in living organisms. There remains a further need for non-invasive techniques for stimulating the production of adult stem cells in living organisms, in particular without introducing chemicals or physically invading the organisms.

### SUMMARY OF THE INVENTION

The present invention meets the needs described above through a method and device for using topically applied acoustical vibrations to impart therapeutic treatments to target tissue of living organisms. More specifically, one or more acoustical transducers are placed directly on the skin of the organism in certain locations, and selected vibration profiles designed to treat specific diseases or conditions are applied to the organism through the transducers. A regimen of regular application of the selected vibration profiles to the specified locations to treat diseases and conditions. This approach is non-invasive and does not involve introducing chemicals or physically invading the organisms. The inventors believe that in certain cases, the therapeutic effect involves the stimulation of the production of adult stem cells in the target tissue areas of the organism.

In a particular embodiment, acoustical vibrations are applied to specific areas of the body in specified pulse profiles that generally include sequences of pulses ranging, for example, from one-half second to three seconds, modulated with an oscillatory signal in the frequency range of 1 Hz to 1500 Hz, and having pulse amplitude in the range of range from about 20 to 5000 microns. The number of application points may vary from one to about thirty, and treatments may be applied once or twice daily over an extended period of weeks, months or years. For example, the acoustical micro-vibration treatments of this type may be applied to the spine, skull, back, pelvis, abdomen, and the upper and low extremities.

For example, the invention may be practiced as a method or a device for stimulating the production of adult stem cells in one or more target tissue areas of a living organism, comprising by placing one or more acoustical transducers on or near the external skin surface overlying or adjacent to the target tissue areas of the living organism. The acoustical transducers produce a vibroacoustic signal profile comprising a sequences of pulses defining a therapeutic regimen configured to impart a therapeutic effect to counteract a specific disease or condition affecting the target tissue areas selected from the group consisting of multiple sclerosis, migraine headache, benign prostatic hyperplasia, spinal cord injury, peripheral neuropathy, Parkinson's disease, and essential tremor. Each pulse has a duration in the range from approximately one-half of a second to approximately ten seconds, an amplitude in the range from approximately 20 microns to approximately 5,000 microns, and adjacent pulses are separated by rest periods in the range from approximately one-tenth of a second to approximately three seconds. The therapeutic regimen includes one or more modulated frequency passes comprising pulses that are modulated with an oscillatory signal in the frequency range from approximately 3 Hz to approximately 1,200 Hz. The therapeutic effect typically involves stimulating the growth of adult stem cells.

For the specific disease or condition of multiple sclerosis, the target tissue areas include a vertebral column area and a head area and the therapeutic regimen includes a first application applied to the vertebral column area having duration between approximately 10 minutes to approximately 60 minutes. The therapeutic regimen also includes second application applied to the head area having duration between approximately 2 minutes to approximately 3 minutes. in particular, the first application may include a plurality of modulated frequency passes, wherein each modulated frequency pass has a duration from approximately 2 minutes to approximately 5 minutes in which each pulse has a duration from approximately 2 seconds to approximately 5 seconds and a modulation frequency from approximately 200 Hz to approximately 1100 Hz. In addition, the second application may include at least one modulated frequency pass including a plurality of pulses in which pulse has a duration from approximately 1 second to approximately 2 seconds, a modulation frequency from approximately 600 Hz to approximately 1100 Hz, and an amplitude of vibration in the range from approximately 20 microns to approximately 1000 microns. The regimen may also include a rest period from approximately 1 minute to approximately 5 minutes between the first application and the second application.

For the specific disease or condition comprises migraine headache, the target tissue areas include head and neck area and the therapeutic regimen includes at least one modulated frequency pass having a duration from approximately 0.5 minutes to approximately 3 minutes in which each pulse has a duration from approximately 0.5 seconds to approximately 2 seconds, a modulation frequency from approximately 700 Hz to approximately 1100 Hz, and an amplitude of vibration in the range from approximately 200 microns to approximately 1000 microns.

For the specific disease or condition comprises benign prostatic hyperplasia, the target tissue areas include a pelvic area and the therapeutic regimen has a duration of approximately 15 minutes to approximately 35 minutes including a plurality of modulated high frequency passes and low frequency passes. Each low frequency pass has a duration of approximately 3 minutes to approximately 5 minutes during which the vibroacoustic signal profile varies in the range from approximately 3 Hz to approximately 100 Hz and each pulse has an amplitude of vibration in the range from approximately 500 microns to approximately 5000 microns. Each high frequency pass has duration from approximately 3 minutes to approximately 5 minutes and includes a plurality of pulses in which each pulse has duration of approximately 2 seconds, a modulation frequency from approximately 400 Hz to approximately 1000 Hz, and amplitude of vibration in the range from approximately 1000 microns to approximately 5000 microns.

For the specific disease or condition of spinal cord injury, the target tissue areas include a vertebral column area and the therapeutic regimen has a duration of approximately 20 minutes to approximately 30 minutes and includes a plurality of modulated frequency passes. Each modulated frequency pass includes a plurality of modulated pulses having amplitude of microvibration from approximately 300 microns to approximately 3000 and a duration from approximately 4 minutes to approximately 6 minutes and comprises a plurality of pulses in which each pulse has a duration from approximately 1 second to approximately 6 seconds and a modulation frequency from approximately 400 Hz to approximately 1200 Hz.

For the specific disease or condition of peripheral neuropathy, the target tissue areas include a nerve affected by peripheral neuropathy and the therapeutic regimen has a duration of approximately 15 minutes to approximately 20 minutes and includes a plurality of modulated high frequency passes and low frequency passes. Each low frequency pass includes a vibroacoustic signal profile in the range from approximately 4 Hz to approximately 40 Hz, amplitude of microvibrations from approximately 500 microns to approximately 4000 microns, and duration from approximately 2 minutes to approximately 3 minutes. Each high frequency pass includes a plurality of pulses in which each pulse includes a modulation frequency from approximately 800 Hz to approximately 1200 Hz, amplitude from approximately 500 microns to approximately 5000 microns, and duration from approximately 0.5 seconds to approximately 1 second. Each high frequency pass has duration from approximately 1 minute to approximately 2 minutes.

For the specific disease or condition of Parkinson's and essential tremor, the target tissue areas include a vertebral column area treatment and a head area treatment. The vertebral column area treatment includes an application comprising a vibroacoustic signal profile in the range from approximately 200 microns to approximately 3000 microns applied to the vertebral column for duration of not more than approximately 15 minutes to 30 minutes. Each modulated frequency pass includes a plurality of pulses in which each pulse includes a modulation frequency from approximately 500 Hz to approximately 1000 Hz and duration from approximately 2 seconds to approximately 7 seconds. The head area treatment includes a vibroacoustic signal profile having amplitude of microvibrations in the range from approximately 100 microns to approximately 1000 microns applied to the head for a duration from approximately 1 minute to approximately 3 minutes, in which each pass has a duration from approximately 1 minute to approximately 3 minutes.

The invention can also be implemented as a method or a device for applying a therapeutic treatment to target tissue areas of a living organism that includes placing one or more acoustical transducers on or near the external skin surface overlying or adjacent to the target tissue areas of the living organism and causing the acoustical transducers to produce a vibroacoustic signal profile including a sequences of pulses defining a therapeutic regimen configured to impart a therapeutic effect to counteract a specific disease or condition affecting the target tissue areas selected from the group consisting of functional constipation, urge incontinence, anemia associated with chronic disease, and renal anemia. Each pulse has duration in the range from approximately 0.5 seconds to approximately 10 seconds, amplitude in the range from approximately 200 microns to approximately 5,000 microns, and adjacent pulses are separated by rest periods in the range from approximately 0.1 seconds to approximately 3 seconds. In addition, the therapeutic regimen includes one or more modulated frequency passes comprising pulses that are modulated with an oscillatory signal in the frequency range from approximately 1 Hz to approximately 1,500 Hz.

For the specific disease or condition comprises functional constipation characterized by colonic inertia, the target tissue areas include an abdominal area and a pelvic area and the therapeutic regimen has a duration of approximately 12 minutes to approximately 15 minutes including a plurality of modulated frequency passes. Each modulated frequency pass includes a plurality of pulses in which each pulse has duration from approximately 4 seconds to approximately 10 seconds and a modulation frequency from approximately 300 Hz to approximately 1500 Hz.

For the specific disease or condition of functional constipation characterized by pelvic floor dysfunction, the target tissue areas include a crotch area and the therapeutic regimen has a duration of approximately 24 minutes to approximately 26 minutes and includes a plurality of high frequency passes and a plurality of low frequency passes. Each high frequency pass a duration from approximately 2.5 minutes to approximately 3 minutes and includes a plurality of pulses in which each pulse has a modulation frequency from approximately 200 Hz to approximately 1500 Hz and duration from approximately 3 seconds to approximately 8 second and an amplitude of approximately 2000 microns to 5000 microns. Each low frequency pass includes a vibroacoustic signal profile in the range from approximately 3 Hz to approximately 200 Hz, amplitude of approximately 2000 microns to 5000 microns, and duration of at least approximately 5 minutes.

For the specific disease or condition of functional constipation characterized by anorectal dysfunction or anismus, the target tissue areas include a crotch area and the therapeutic regimen has a duration from approximately 20 minutes to approximately 22 minutes and includes a plurality of modulated frequency passes. Each modulated frequency pass has duration of approximately 2 minutes and includes a plurality of pulses in which each pulse has a modulation frequency from approximately 400 Hz to approximately 1500 Hz and amplitude from approximately 100 microns to approximately 2000 microns.

For the specific disease or condition comprises urinary urge incontinence, the target tissue areas include a low abdominal and pelvic area and the therapeutic regimen has a duration of from approximately 18 minutes to approximately 22 minutes and comprises a plurality of high frequency passes and one or more low frequency passes. Each high frequency pass has duration from approximately 3 minutes to approximately 4 minutes and includes a plurality of pulses in which each pulse has a modulation frequency from approximately 200 Hz to approximately 1500 Hz and amplitude from approximately 100 microns to approximately 500 microns. Each low frequency pass includes a vibroacoustic signal profile in the range from approximately 3 Hz to approximately 200 Hz, duration from approximately 1 minute to approximately 3 minutes, and amplitude from approximately 200 microns to approximately 2000 microns.

For the specific disease or condition of anemia associated with chronic disease or renal anemia, the target tissue areas include a vertebral column area and pelvic area and the therapeutic regimen has a duration of approximately 15 minutes to approximately 30 minutes and comprises a plurality of modulated frequency passes. For this therapy, each modulated frequency pass comprises a plurality of non-modulated pulses having amplitude of microvibration from approximately 300 microns to approximately 4000, and each modulated frequency pass has a duration from approximately 2 to approximately 5 minutes and comprises a plurality of pulses, each pulse having a duration from approximately 2 second to approximately 10 seconds and comprising a modulation frequency from approximately 400 Hz to approximately 1300 Hz.

For the specific target tissue areas including the vertebral column area and pelvic area, the therapeutic regimen has duration of approximately 15 minutes to approximately 30 minutes and comprises a plurality of modulated frequency passes; Each modulated frequency pass comprises a plurality of non-modulated pulses having amplitude of microvibration from approximately 300 microns to approximately 4000; and each modulated frequency pass has a duration from approximately 2 to approximately 5 minutes and comprises a plurality of pulses, each pulse having a duration from approximately 2 second to approximately 10 seconds and comprising a modulation frequency from approximately 400 Hz to approximately 1300 Hz.

### BRIEF DESCRIPTION OF THE DRAWING

FIG. 1 is a front view of a micro-vibration unit suitable for implementing the present invention.

FIG. 2 is a graphical representation of a first micro-vibration profile that may be applied through the micro-vibration unit to a living organism to stimulate the production of adult stem cells in the organism.

FIG. 3 is a graphical representation of a second micro-vibration profile that may be applied through the micro-vibration unit to a living organism to stimulate the production of adult stem cells in the organism.

FIG. 4 is a graphical representation of a third micro-vibration profile that may be applied through the micro-vibration unit to a living organism to stimulate the production of adult stem cells in the organism.

FIG. 5 is a graphical representation of a fourth micro-vibration profile that may be applied through the micro-vibration unit to a living organism to stimulate the production of adult stem cells in the organism.

FIG. 6 is a graphical representation of a fifth micro-vibration profile that may be applied through the micro-vibration unit to a living organism to stimulate the production of adult stem cells in the organism.

FIG. 7A is a graphical representation of an area overlying the thoracic spine of a human selected for micro-vibration treatment.

FIG. 7B is a graphical representation of specific points within the area of FIG. 7A for applying micro-vibration treatments to stimulate the production of adult stem cells.

FIG. 8A is a graphical representation of an area overlying the front portion of the cranium of a human selected for micro-vibration treatment.

FIG. 8B is a graphical representation of specific points within the area of FIG. 8A for applying micro-vibration treatments to stimulate the production of adult stem cells.

FIG. 9A is a graphical representation of an area overlying the rear portion of the cranium of a human selected for micro-vibration treatment.

FIG. 9B is a graphical representation of specific points within the area of FIG. 9A for applying micro-vibration treatments to stimulate the production of adult stem cells.

FIG. 10A is a graphical representation of an area overlying the lower spine of a human selected for micro-vibration treatment.

FIG. 10B is a graphical representation of specific points within the area of FIG. 10A for applying micro-vibration treatments to stimulate the production of adult stem cells.

FIG. 11A is a graphical representation of areas overlying the shoulder blades of a human selected for micro-vibration treatment.

FIG. 11B is a graphical representation of specific points within the areas of FIG. 11A for applying micro-vibration treatments to stimulate the production of adult stem cells.

FIG. 12A is a graphical representation of areas overlying the lower back of a human selected for micro-vibration treatment.

FIG. 12B is a graphical representation of specific points within the areas of FIG. 12A for applying micro-vibration treatments to stimulate the production of adult stem cells.

FIG. 13A is a graphical representation of an area overlying a front portion of the abdomen of a human selected for micro-vibration treatment.

FIG. 13B is a graphical representation of specific points within the area of FIG. 13A for applying micro-vibration treatments to stimulate the production of adult stem cells.

FIG. 14A is a graphical representation of an area overlying a rear portion of the abdomen of a human selected for micro-vibration treatment.

FIG. 14B is a graphical representation of specific points within the area of FIG. 14A for applying micro-vibration treatments to stimulate the production of adult stem cells.

FIG. 15A is a graphical representation of areas overlying the leg muscles of a human selected for micro-vibration treatment.

FIG. 15B is a graphical representation of specific points within the area of FIG. 15A for applying micro-vibration treatments to stimulate the production of adult stem cells.

FIG. 16A is a graphical representation of areas overlying the inner arm muscles of a human selected for micro-vibration treatment.

FIG. 16B is a graphical representation of specific points within the area of FIG. 16A for applying micro-vibration treatments to stimulate the production of adult stem cells.

FIG. 17A is a graphical representation of areas overlying the outer arm muscles of a human selected for micro-vibration treatment.

FIG. 17B is a graphical representation of specific points within the area of FIG. 17A for applying micro-vibration treatments to stimulate the production of adult stem cells.

FIG. 18 is a graphical representation of specific points for applying micro-vibration treatments to stimulate the calf muscle.

FIG. 19 is a graphical representation of specific points for applying micro-vibration treatments to stimulate the thigh muscle.

FIG. 20 is a conceptual illustration of a pad for holding micro-vibration transducers in place for applying treatment to the back.

FIG. 21 is a conceptual illustration of a pad for holding micro-vibration transducers in place for applying treatment to the head and neck.

FIG. 22 is a conceptual illustration of a pad attached to a chair holding micro-vibration transducers in place for applying treatment to the back.

FIG. 23 is a conceptual illustration of a pad attached to a chair holding micro-vibration transducers in place for applying treatment to the back and seat.

FIG. 24 is a conceptual illustration of a pad attached to a chair holding micro-vibration transducers in place in an alternative configuration for applying treatment to the back.

FIG. 25 is a conceptual illustration of a user sitting in a chair having an attached pad holding micro-vibration transducers in place for applying treatment to the back.

FIG. 26 is a conceptual illustration of a knee wrap holding micro-vibration transducers in place for applying treatment to the knee.

FIG. 27 is a conceptual illustration of an elbow wrap holding micro-vibration transducers in place for applying treatment to the elbow.

FIG. 28 is a conceptual illustration of a head wrap holding micro-vibration transducers in place for applying treatment to the head.

FIG. 29 is a conceptual illustration of a multi-purpose strap holding micro-vibration transducers in place for applying treatment to desired areas.

### DETAILED DESCRIPTION OF THE EMBODIMENTS

Mechano-transduction is the process by which cells in living organisms convert mechanical stimuli into biochemical signals. The inventors have discovered that cells react to acoustical micro-vibration stimuli by trying to protect tissue integrity, which stimulates the production of adult stem cells in the tissue. For example, it is believed sound energy stimulates chondrocyte, which leads to enhance proteoglycan synthesis, and ultimately results in augment synovial fluid production and cartilage repair. It is also believed that acoustical micro-vibrational stimulation enhances chondrocyte proliferation in living tissue, especially through the application of repetitive pulses with oscillatory waveform in the frequency range from 1 Hz to 1500 Hz. Analysis of the test data further suggests that genetically coded chondral growth is up regulated by these mechanical signals. Other cell types that are believed to respond to this type of mechanical stimuli with increased production of adult stem cells include osteocyte, myocardiocyte, monocyte, and endothelium.

Adult stem cells can differentiate in all of above mentioned cell types and serve as the precursors for tissue repair. The primary function of adult stem cells is to maintain and repair tissues wherever it is found. The proliferation of adult stem cells increases when tissue is damaged or stimulated in a manner that mimics the effect of damaged. Known factors that increase adult stem cell production include chemical substances (such as growth factors) and hypoxemia (decreased concentration of oxygen in the tissue). In accordance with the present invention, adult stem cell production is stimulated by mechanical stimuli in the form of acoustical micro vibrations. The adult stem cells respond to the micro vibration treatment by multiplying faster in the area where the treatment is applied. This action is similar to chondrocyte response to increase synthesis of proteoglycan to protect cartilage or osteocyte to produce more bone in response to mechanical stress.

Vibroacoustic stimulation has proven to be beneficial for treating multiple sclerosis, migraine headache, benign prostatic hyperplasia, spinal cord injury, peripheral neuropathy, Parkinson's disease, essential tremor, anemia associated with chronic disease, and renal anemia. Although the inventors have concluded that the stimulation of the growth of adult stem cells plays an important role in treating these diseases and conditions, the therapeutic effect has been established regardless of whether increased stem cell production is involved in the improvement of the disease or condition. In addition, functional constipation and urge incontinence are improved without any identified contribution from increased stem cell production. Accordingly, it should be appreciates that specific vibroacoustic treatment regimens described in this specification have demonstrated therapeutic effects for multiple sclerosis, migraine headache, benign prostatic hyperplasia, spinal cord injury, peripheral neuropathy, Parkinson's disease, essential tremor, functional constipation, urge incontinence, anemia associated with chronic disease and renal anemia, which in certain cases apparently is believed to involve the stimulation of adult stem cell production.

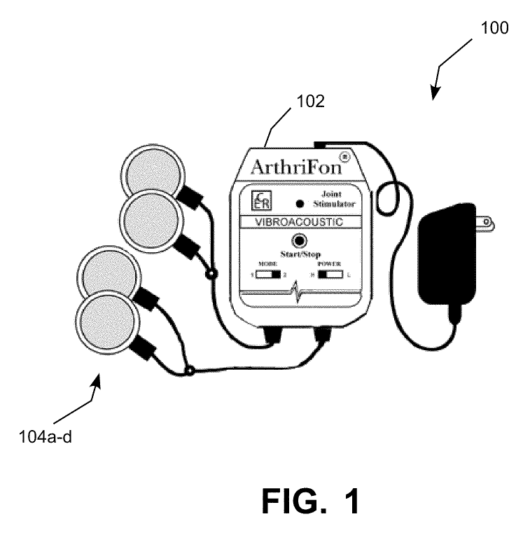

FIG. 1 is a front view of a micro-vibration unit 100 suitable for implementing the present invention. The micro-vibration unit includes a control unit 102 and a plurality of transducers 104a-d that convert electric drive signals into acoustical pulse waves. The transducers are configured to be applies directly to the skin, for example with tape, elastic straps or other suitable attachment devices. A prior but similar micro-vibration unit is described in commonly owned U.S. patent application Ser. No. 10/761,726 (Publication No. 2004-0167446), which is incorporated herein by reference. The micro-vibration unit 100 is different from the prior unit in that the new unit 100 is configured to produce the pulse wave profiles described below, which have been found to be effective for stimulating adult stem cell production in living organism.

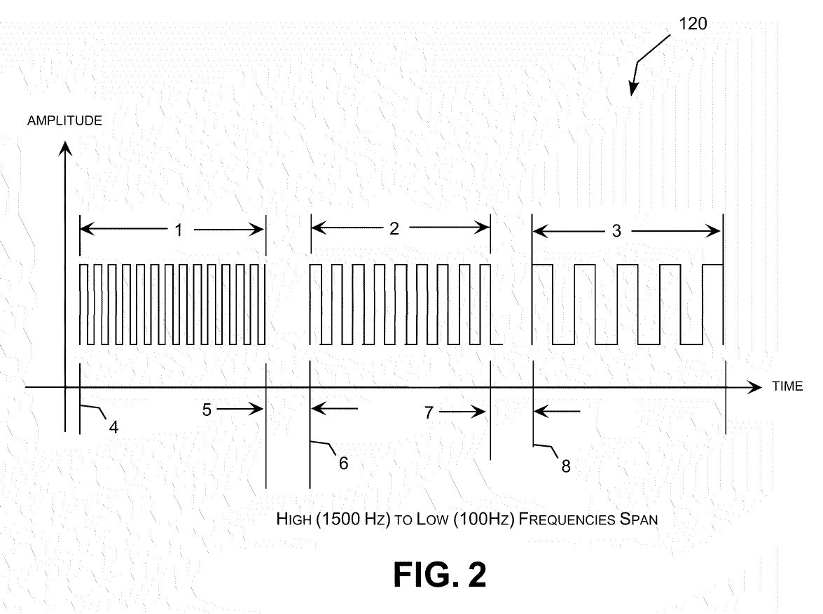

FIG. 2 is a graphical representation of an illustrative portion of a micro-vibration profile 120 that may be applied through the micro-vibration unit 100 to a living organism to stimulate the production of adult stem cells in the organism. The pulse wave profile 120 includes three pulses indicated as pulse 1, pulse 2 and pulse 3. Although only three pulses are shown, the full pulse wave profile 120 may include many more pulses, such as tens or even hundreds of pulses depending on the length of the treatment. Each pulse (illustrated by pulses 1, 2, and 3) is typically in the range of one-half to ten seconds in duration, and the time between pulses (illustrated by pulse separation periods 5 and 7) and 3) is typically in the range of one-tenth to three seconds. In addition, the modulation frequency of the pulses typically varies from pulse to pulse within the range of 1500 Hz to 100 Hz. As shown conceptually in FIG. 2, the modulation frequency may decrease with each successive pulse. For example, the pulse profile may begin at 1500 Hz, step down with pulse-to-pulse increments of 100 Hz, and end with a final pulse 100 Hz. Of course, this is relatively simple pulse profile provided to illustrate the technique, and many variations may be implemented.

The inventors believe that the leading edges 4, 6 and 8 of micro-vibration pulses within the indicated frequency range have the effect of expanding capillaries in the tissues underlying the area of the treatment, and the cessation of the pulses relaxes the capillaries. Therefore, repeated application of the pulse illustrated by pulses 1, 2, and 3 has the effect of repeatedly expanding and relaxing the capillaries. The repeated expansion and contraction of the capillaries is believed to have the effect of increasing the delivery of nutrition and oxygen, which has the effect of stimulating the production of adult stem cells in the affected tissue.

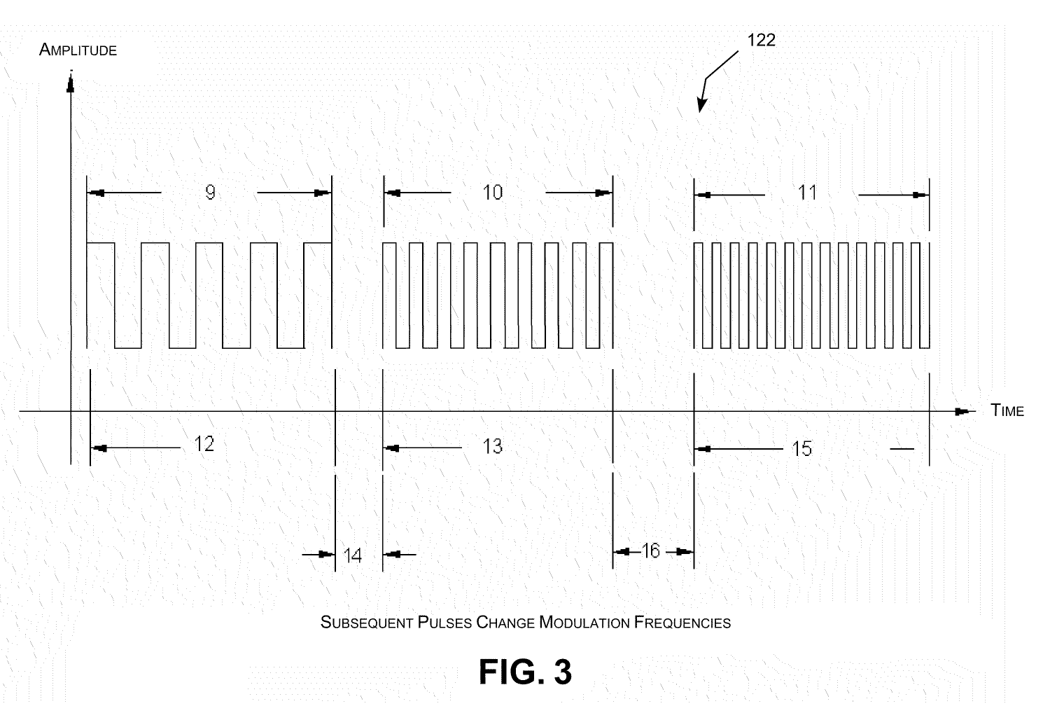

FIG. 3 is a graphical representation of a micro-vibration profile 122 that is similar to the profile 120, except that it starts at the low frequency end of the range at about 100 Hz, increases in increments of about 100 Hz up to the upper end of the range at about 1500 Hz. Again, each pulses typically has a duration in the range of one-half to ten seconds, and the pulse separation time is typically in the range of one-tenth of a second to three seconds.

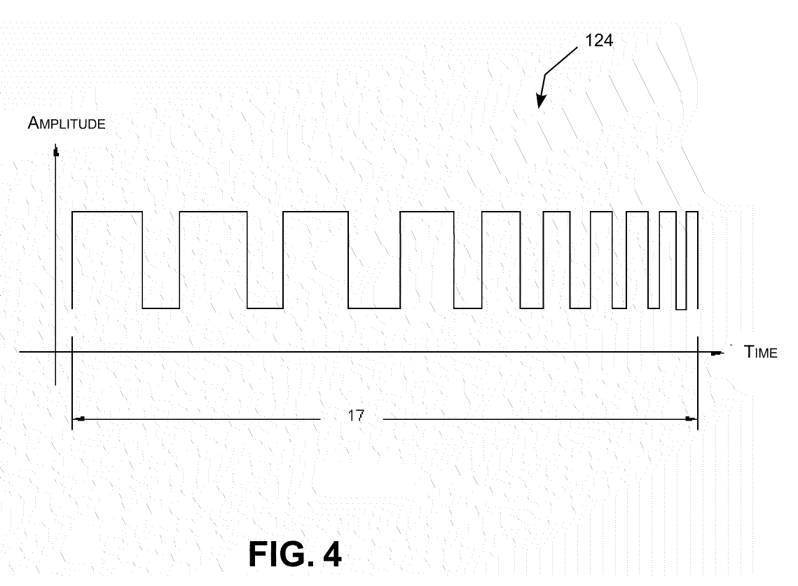

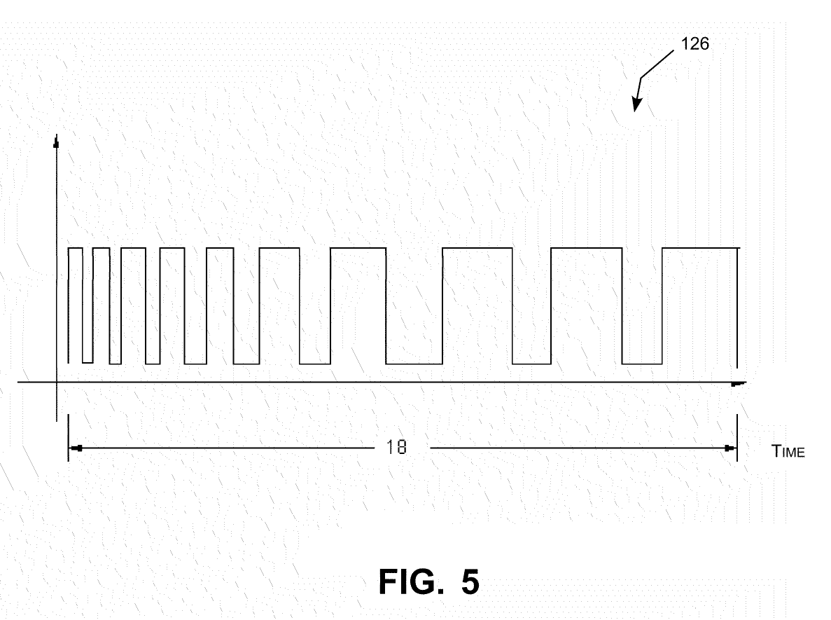

FIG. 4 is a graphical representation of a micro-vibration pulse profile 124 that may be applied through the micro-vibration unit 100 to a living organism to stimulate the production of adult stem cells in the organism. This micro-vibration profile 124 begins with at a very low frequency of about 1 Hz and builds up to about 120 Hz. The duration of the pulse profile 124 can vary from about 30 second to 30 minutes, and can be applied repetitively, as desired. This type of ultra-low frequency treatment is typically applied for three minutes to one hour, and has been found to be suitable for stimulating the production of adult stem cells in muscle and tendon tissue.

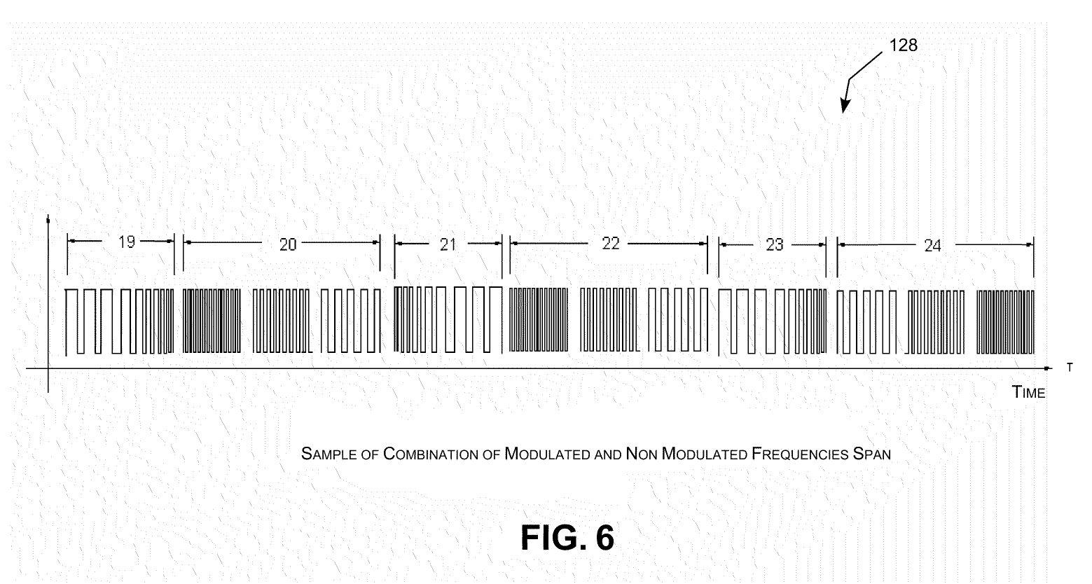

FIG. 6 is a graphical representation of a micro-vibration profile 126 that is similar to the profile 124, except that starts with at the high frequency end of the range at about 120 Hz, decreases down to the lower end of the range at about 1 Hz. Like the profile 124, the profile 126 can vary from about 30 second to 30 minutes, and can be applied repetitively, as desired.

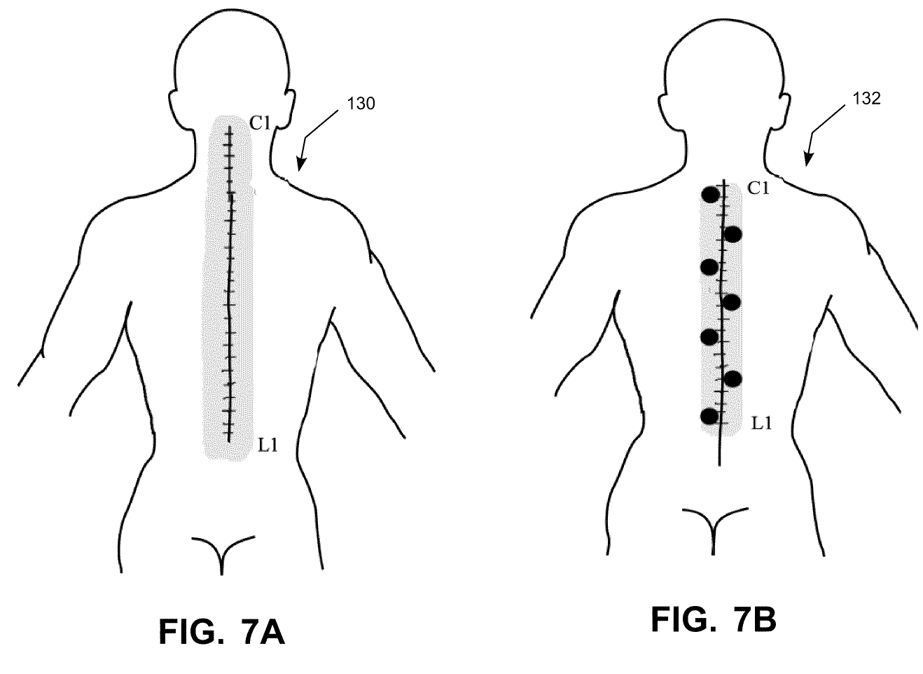

FIG. 7 is a graphical representation of a micro-vibration profile 128 that is a combination of the profiles 120, 122, 124 and 126 described above. The inventors have found that a consistent regimen of applying this type of profile once or twice daily over an extended period, such as several months, has the desired effect of stimulating the production of adult stem cells in the a range of tissues, such as muscle, tendon, fat, liver and bone marrow. Of course, the particular profile 128 shown in merely illustrative, and alternative pulse shapes, frequencies, durations and combinations can be applied using the present invention. Nevertheless, it should be appreciated that the profile 128 within the parameters described above has been found to be effective profile for practicing the present invention.

FIG. 7A is a graphical representation of an area 130 overlying the thoracic spine of a human, and FIG. 7B shows specific points 132 for applying micro-vibration treatments to stimulate the production of adult stem cells within the tissues underlying area 130. The tissues underlying area 130 include a large portion of the spinal cord, bone marrow, skeletal muscles and fat tissue located along vertebral column staring from C1 vertebra down to the L1 vertebra, and approximately three inches wide along both sides of the vertebral midline.

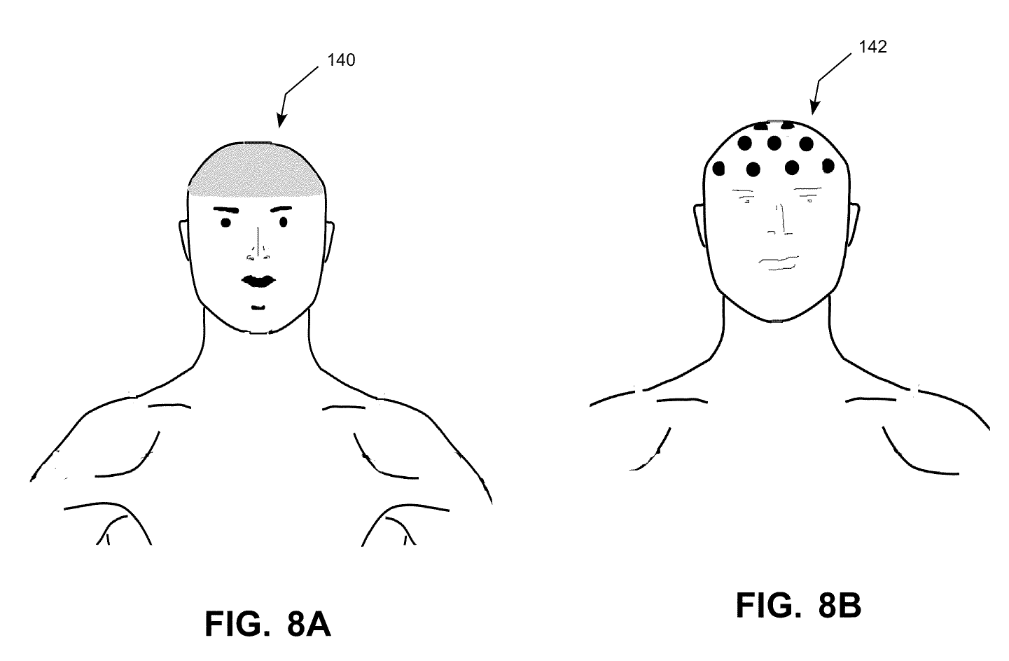

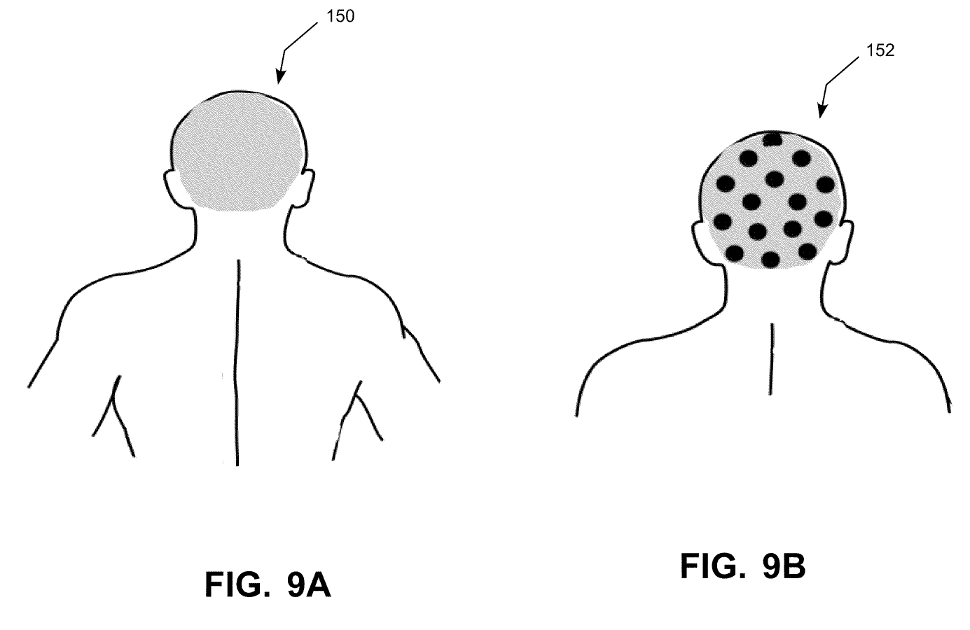

FIG. 8A is a graphical representation of an area 140 overlying the front portion of the cranium of a human, and FIG. 8B shows specific points 142 for applying micro-vibration treatments to stimulate the production of adult stem cells within the tissues underlying area 140. FIG. 9A is a graphical representation of an area 150 overlying the front portion of the cranium of a human, and FIG. 9B shows specific points 152 for applying micro-vibration treatments to stimulate the production of adult stem cells within the tissues underlying area 150. The tissues underlying the areas 140 and 150 include the brain and bone marrow located on the skull.

FIG. 10A is a graphical representation of an area 160 overlying the lower spinal area of a human, and FIG. 10B shows specific points 162 for applying micro-vibration treatments to stimulate the production of adult stem cells within the tissues underlying area 160. The tissues underlying the area 160 includes the spine, bone morrow, skeletal muscles and fat tissue located along vertebral column staring from L1 vertebral body down to S5 vertebral body and approximately three inches to both sides of the vertebral midline.

FIG. 11A is a graphical representation of an area 170 overlying the lower back area of a human, and FIG. 11B shows specific points 172 for applying micro-vibration treatments to stimulate the production of adult stem cells within the tissues underlying area 170. The tissues underlying the area 170 include the bone morrow, skeletal muscles and fat tissue located in the region of both scapulas.

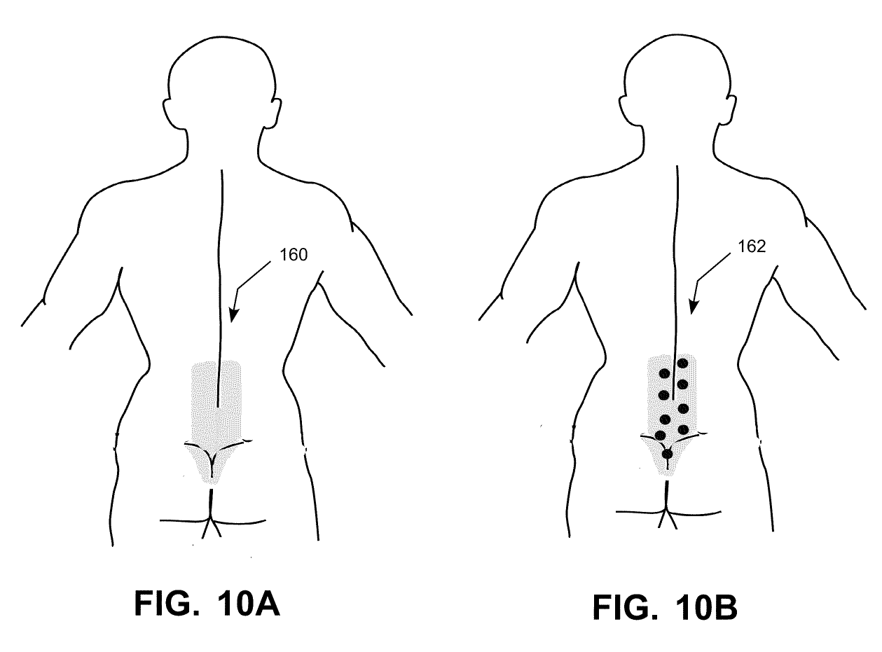

FIG. 12A is a graphical representation of an area 180 overlying the lower back area of a human, and FIG. 12B shows specific points 182 for applying micro-vibration treatments to stimulate the production of adult stem cells within the tissues underlying area 180. The tissues underlying the area 180 include the bone morrow, skeletal muscles and fat tissue located in region of the flat bones of pelvis.

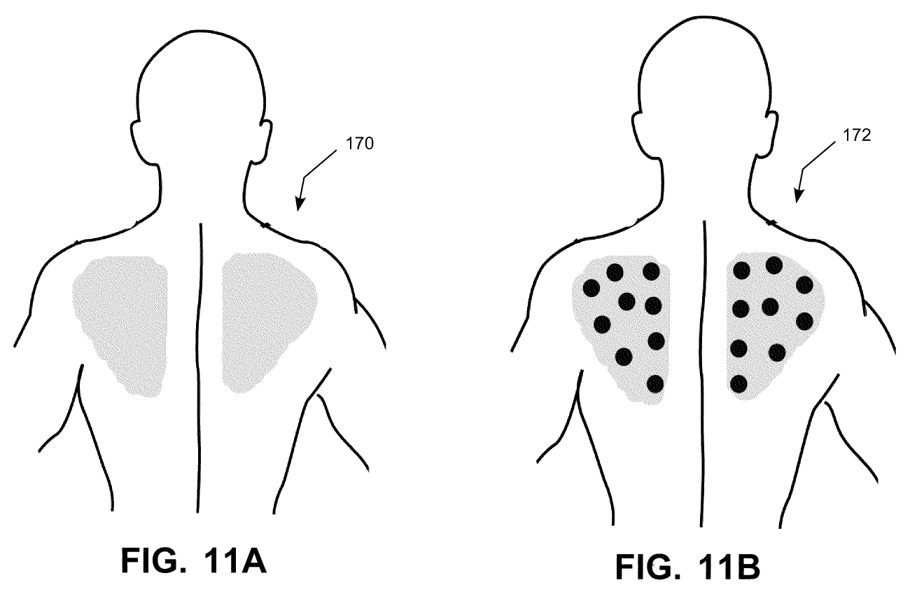

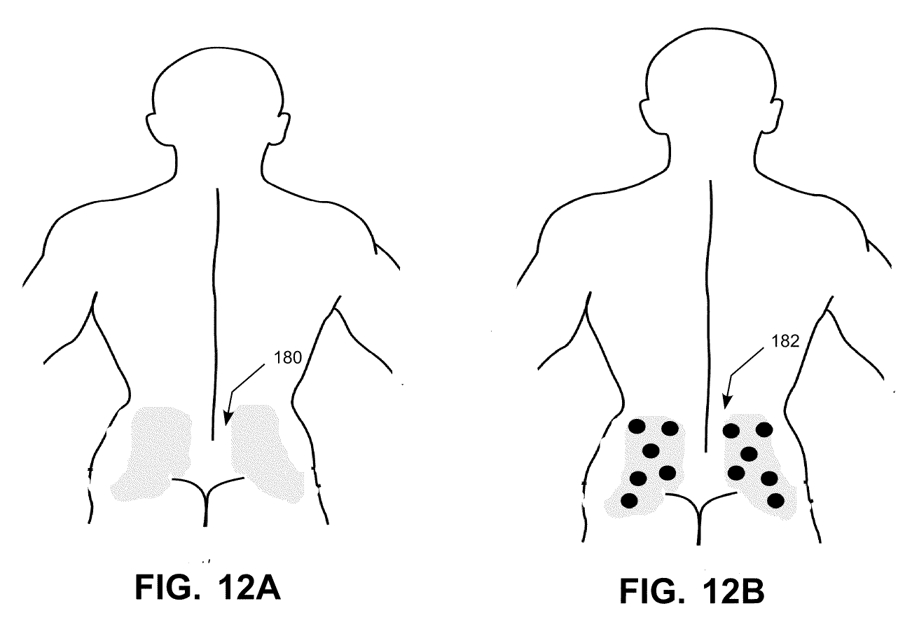

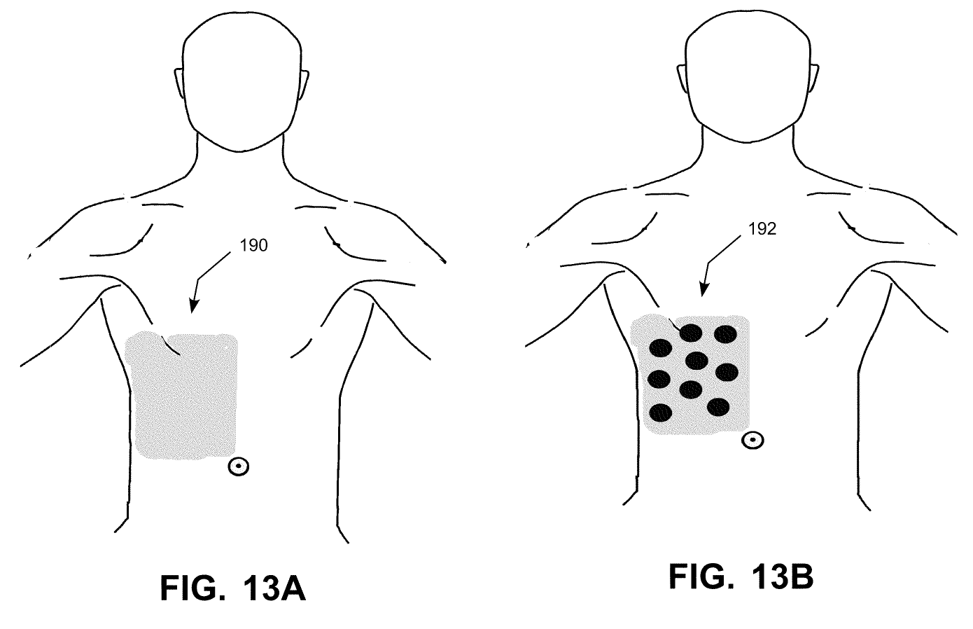

FIG. 13A is a graphical representation of an area 190 overlying a front portion of the abdomen of a human, and FIG. 13B shows specific points 192 for applying micro-vibration treatments to stimulate the production of adult stem cells within the tissues underlying area 190. FIG. 14A is a graphical representation of an area 194 overlying a rear portion of the abdomen of a human, and FIG. 14B shows specific points 196 for applying micro-vibration treatments to stimulate the production of adult stem cells within the tissues underlying area 194. The tissues underlying the areas 190 and 194 include the liver, the skeletal muscles and fat tissue in the region of the liver.

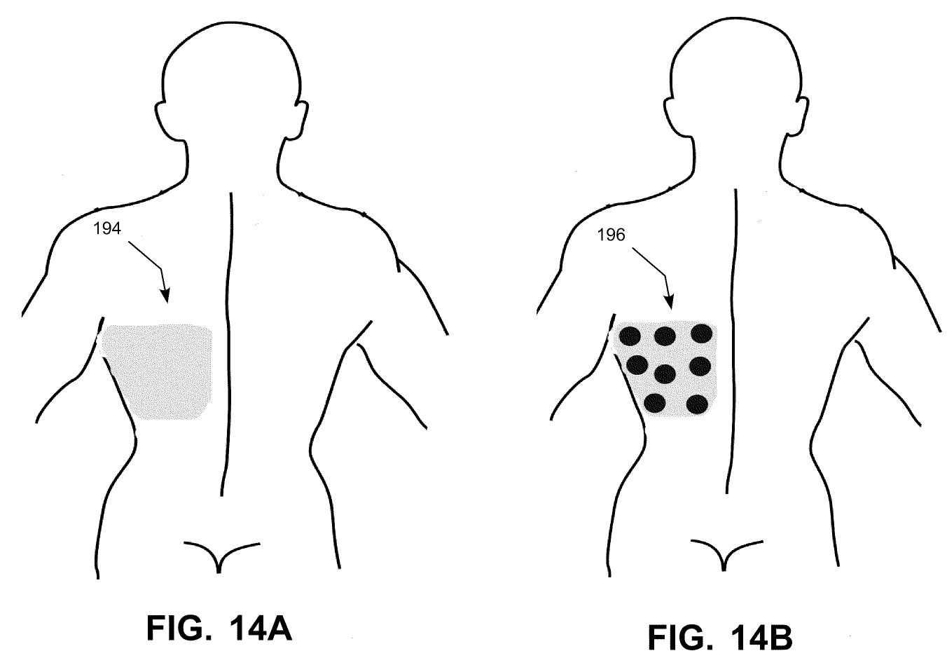

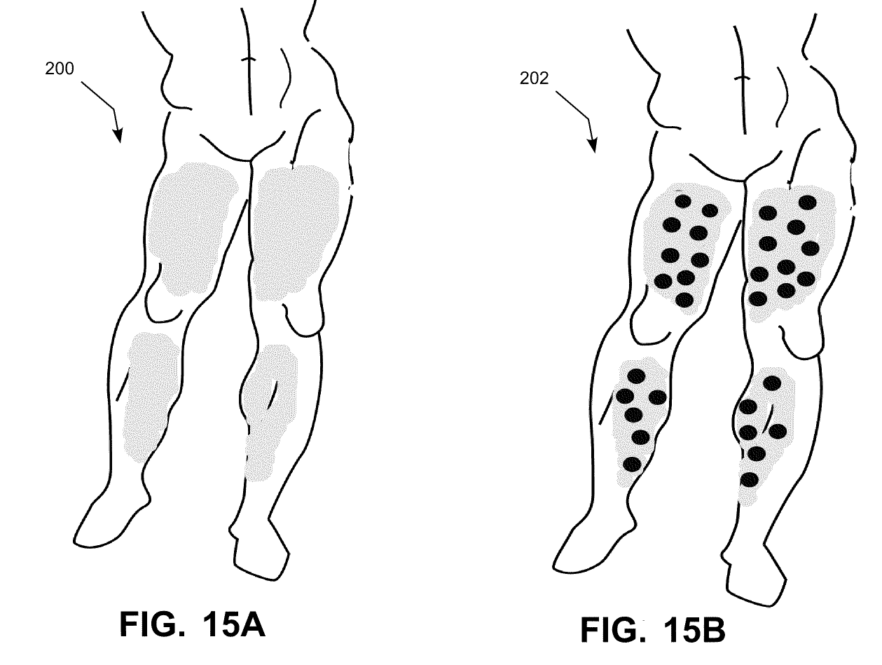

FIG. 15A is a graphical representation of an area 200 overlying the leg area of a human, and FIG. 15B shows specific points 202 for applying micro-vibration treatments to stimulate the production of adult stem cells within the tissues underlying area 200. The tissues underlying the area 180 include the bone morrow, skeletal muscles and fat tissue located in region of the leg muscles.

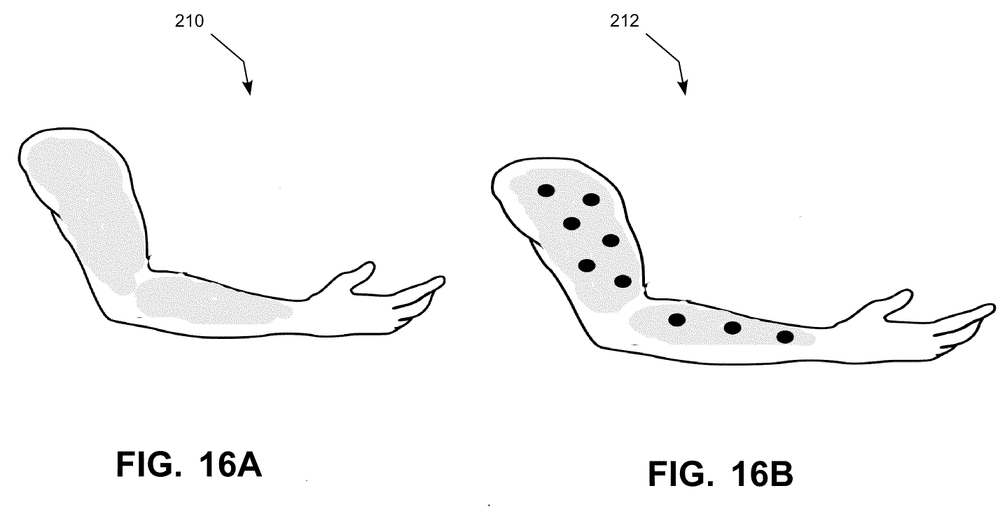

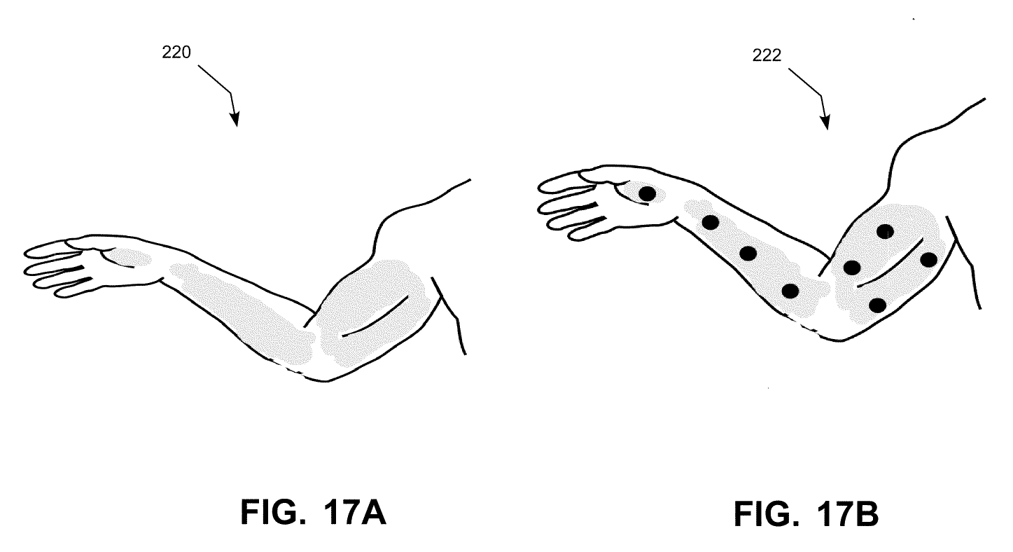

FIG. 16A is a graphical representation of an area 210 overlying the inner arm area of a human, and FIG. 16B shows specific points 212 for applying micro-vibration treatments to stimulate the production of adult stem cells within the tissues underlying area 210. FIG. 17A is a graphical representation of an area 220 overlying outer arm of a human, and FIG. 17B shows specific points 222 for applying micro-vibration treatments to stimulate the production of adult stem cells within the tissues underlying area 220. The tissues underlying the areas 210 and 220 include the bone morrow, skeletal muscles and fat tissue located in region of the arm muscles.

The term “frequency pass” refers to a number of modulated or non-modulated pulses applied by the vibroacoustic device. A modulated pulse is a low frequency pulse filled with higher frequency pulses. The pulses may have any desired shape, such as sinusoidal, rectangular, triangular, and so forth. The modulation frequency may be constant during a pulse (i.e., constant frequency pulse) or the frequency may vary during a pulse (i.e., variable frequency pulse). The modulation frequency may be the same for every pulse (i.e., constant frequency pulse sequence) or the modulation frequency may vary from pulse to pulse (i.e., variable frequency pulse sequence). The amplitude of the micro-vibration signal may also vary within a pulse or from pulse to pulse. A non-modulated pulse is a pulse in which a constant DC value is applied by the vibroacoustic transducer. A rest period is a time between pulses when the vibroacoustic transducers apply no stimulation.

During a pulse sequence, the pulses may have the same duration (i.e., constant duration pulses) or the duration may vary from pulse to pulse (i.e., variable duration pulses). In addition, the rest period between pulses may remain constant or it may vary from rest period to rest period. A pulse sequence in which the duration of the pulses and the rest periods between the pulses remains the same is referred to as a constant pulse.

The parameters of a pulse sequence, such as the frequency, amplitude, duration, and number of pulses of the modulated or non-modulated pulses can all be varied to produce different pulse sequences. The term High Frequency (HF) pass refers to a pulse sequence that includes a number of modulated pulses. Unless otherwise notes, the HF pass includes pulses with constant pulse duration and amplitude, where frequency and amplitude change from pulse to pulse within the pass. For example, if modulated frequency starts from 1200 Hz in the pulse #1 and duration of that pulse is 2 seconds the next pulse #2 may have modulated frequency of 1995 Hz and duration of that pulse can be 2.01 seconds to keep same number of cycles for each HF pulse. The amplitude of each pulse in a HF pass may also vary from maximum to minimum during the HF pass, typically from 50 to 1000 microns. The rest time between pulses during the HF pass ranges from 0.01 to 0.1 seconds unless a different value is specified.

The term Low Frequency (LF) pass refers to a pulse sequence that includes non-modulated pulses where frequency and amplitude of the stimulation change smoothly from the beginning to the end of the LF pass. The duration the LF pass varies from 0.5 second to 5 seconds. The frequency applied during the LF pass typically varies from 0.5 Hz to 120 Hz. The term Fixed Frequency (FF) pass or period refers to a period during which the vibroacoustic transducer applies a constant frequency.

Specific micro-vibration treatment regimen have been developed to reduce symptoms, repair tissue and effect cures for a number of diseases and conditions. The specific treatment regimens can be applied daily or several times per day for as long as the therapeutic effect is desired, typically an extended period of weeks, months or years.

Multiple sclerosis. Today, multiple sclerosis is recognized as a chronic, inflammatory, demyelinating autoimmune disease of the Central nervous system (CNS). The disease is characterized by damage to the myelin covering nerve cells and damage to the underlying nerve cell fibers, which leads to slowed or blocked transmission of signals by the nerve cells. The nerve damage causes reduced or lost muscle function. Vibroacoustic stimulation helps to repair the nerve damage caused by multiple sclerosis by stimulating the production of adult stem cells, which repair the nerve cells and the myelin covering nerve cells. The stem cells repair oligodenrocytes, improve nerve cell conduction, open capillaries to provide better blood circulation, and produce an anti-inflammatory effect.

The vibroacoustic treatment regimen for multiple sclerosis includes a first application applied to the spinal cord, followed by a rest period, followed by a second application applied to the head. The spinal cord application includes a number HF passes, LF passes and FF periods applied to the spinal cord lasting from 30 to 60 minutes. After a rest period of 60 minutes, the second application lasting from 2 to 3 minutes is applied to the head.

The spinal cord application uses vibroacoustic stimulation for treatment of multiple sclerosis lesions located in spinal cord to achieve micro-vibration in the sound frequencies to the spinal cord. The transducers are placed along the vertebral column staring from C1 vertebral body down to L1 vertebral body and 3 inches wide to the right and 3 inches wide to the left from vertebral midline as shown in FIG. 7B.

The spinal cord application includes a number of HF and LF passes followed by a 3-minute FF period during which a constant frequency in the range of 350 and 450 Hz is applied. The spinal cord application begins with two HF passes; followed by one LF pass, followed by two more HF passes, and concludes with one FF period. This sequence of passes can be repeated, as desired. The spinal cord application typically includes one sequence (2 HF passes, LF pass, 2 HF passes, one FF period) lasting about 30 minutes, which can be repeated to produce a total first application lasting 60 minutes.

During the HF pass, each pulse has a duration ranging from 2 to 5 seconds and typically applies a constant frequency during each pulse. The frequency and amplitude typically varies from pulse to pulse during the HF pass. The modulation frequency changes from pulse to pulse from 1100 Hz to 800 Hz or from 800 Hz to 1100 Hz over the pulse sequence. The pulse-to-pulse change in frequency can be selected produce the desired duration for the HF pass. There are a number of parameters that can be changed from pulse to pulse, as desired, including the pulse duration, rest time, and modulation frequency. The duration of the HF pass last from 3 to 5 minutes. The duration of the LF pass is typically from 1 to 5 minutes. During the LF pass, the signal applied by the Vibroacoustical transducer varies from smoothly from 1 Hz to 120 Hz or from 120 Hz to 1 Hz over the course of the pass. The total number of HF and LF passes during the first application ranges from 5 to 10 (average 3 min per pass) plus the fixed frequency (FF) interval. The duration of the entire application should not be less than 30 minutes or longer than 60 minutes. The recommended duration for the application is from 30 to 60 minutes

After a rest period of about an hour, the second application is applied to the head as shown in FIGS. 8B and 9B. The head application includes one HF pass in which the frequency varies from pulse to pulse starting from 1100 Hz and ending with 900 Hz or in reverse order from 900 Hz to 1100 Hz. The duration of each pulse can vary from 1.5 seconds to 2 seconds. The rest time between each modulated pulse during the HF pass can vary and should be not less than about 0.2 seconds. The number of modulated pulses is from 30 to 60 pulses. The duration of the head application is from 2 to 3 minutes.

Migraine headache. Researchers believe that migraine headaches may be caused by functional changes in the trigeminal nerve system, which is a major pain pathway in your nervous system, and, by imbalances in brain chemicals, including serotonin, which plays a regulatory role for pain messages going through this pathway. During a migraine headache, serotonin levels drop. Researchers believe this causes the trigeminal nerve to release substances called neuropeptides, which travel to the brain's outer covering known as the meninges. There the neuropeptides cause blood vessels to become dilated and inflamed. The result is a migraine headache pain.

The Vibroacoustic stimulation for treatment for migraine headache involves transmitting micro-vibration in the sound frequencies to the brain and meningeal membranes. The vibroacoustic treatment stimulate the production of adult stem cells that repair never cells, improve nerve cell fiber conduction, and provide better blood circulation in the trigeminal nerve system.

The treatment regimen for migraine headache includes one HF passes applied to the head as shown in FIGS. 8B and 9B. During the HF pass, this modulation frequencies applied start from 1100 Hz and end with frequency of 700 Hz or in reverse order from 700 Hz to 1100 Hz. The duration of each pulse can vary from 1 seconds to 2 seconds. The number of modulated pulses is from 30 to 60 pulses. The duration of the application is from 1 to 2 minutes. This treatment regimen can be applied daily or several times per day for as long as the therapeutic effect is desired, typically an extended period of weeks, months or years.

Benign Prostatic Hypertrophy (BPH). It is common for the prostate gland to become enlarged as a man ages. Doctors call this condition benign prostatic hyperplasia (BPH), or benign prostatic hypertrophy. The micro-vibration treatment regimen for BPH includes vibroacoustic stimulation to suppress alpha-sympathetic nervous system to cause bladder neck relaxation to improve urea flow and decrease prostate volume by improving blood circulation in the relevant area. Vibroacoustic stimulation for treatment of BPH is designed to achieve transmission of micro-vibration in the sound frequencies to the prostate. The vibroacoustic transducers are located in a first area about 5 inches above ramie pubis (pelvic bone in front) and 8 inches wide to the right and 8 inches wide to the left from abdominal midline. Additional transducers can also be placed in a second area from the base of the penis to the anus about two inches wide to the right and 2 inches wide to the left from pelvic midline. The treatment regimen can be applied simultaneously to the first and second areas, or it can be applied to each area in separate treatments.

The treatment regimen for BPH includes a number HF passes, LF passes and a FF period with modulation at 400 Hz at the end of application. The total application time is from 30 to 35 minutes. The application starts with three LF passes 3 to 5 minutes long separated by rest periods of 10 seconds. The LF passes are followed by two HF passes 3 to 5 minutes long separated by a rest period of 10 seconds. This is followed by another LF pass 3 to 5 minutes in duration, followed by 5 to 10 pulses about one second in duration each with modulation frequency of 400 Hz. Each frequency of the vibroacoustic stimulation applied during the LF pass varies from 3 Hz to 100 Hz. The duration of each LF pass is no less than 3 minutes. The frequency of LF smoothly changes from 3 Hz to 100 Hz.

The High frequency pass consists of pulses with duration of 2 seconds and modulation starting at 1200 Hz and change to conclude the HF pass at 600 Hz.

Spinal cord injury. The treatment regimen for spinal cord injury will use vibroacoustic stimulation to increase the production of adult stem cells to repair glial cells and neurons in the spinal cord and improve nerve cell fiber conduction. The transducers are placed near the spinal cord in the area of the injury, for example as shown in FIG. 10B. The application can also be applied along vertebral column staring from C1 vertebral body down to L1 vertebral body and 3 inches wide to the right and to the left from vertebral midline or direct on the vertebral column as shown in FIG. 7B.

The treatment regimen consists of number HF and LF passes followed by 5 one-second FF periods with modulation at 400 Hz at the end of application. The sequence of HF and LF passes is as follows: 2 HF passes followed by 2 LF passes. After those four passes, there will be a 10 second rest period. After the rest period, 3 HF passes followed by 1 LF pass. Thereafter there will be an additional 5-second FF period with modulation at 400 Hz at the end of application. During the HF pass, each pulse has constant frequency and amplitude, and the frequency and amplitude vary from pulse to pulse. The duration of each HF pass is from 3 to 5 minutes, the duration of each HF pulse varies from 1 to 2 seconds, and the modulation frequency varies from 1200 Hz to the 400 Hz or from 400 HZ to the 1200 Hz over the course of the HF pass. The rest time between HF modulated pulses is from 0.1 to 0.2 seconds. The amplitude of the micro-vibration varies from 50 microns to 1000 microns. The amplitude of LF non-modulated pulses varies from 300 to 2000 microns. The total application time is from 24 to 28 minutes.

Peripheral neuropathy. Peripheral neuropathy is a problem with the nerves that carry information to and from the brain and spinal cord. This produces pain, loss of sensation, and inability to control muscles. The treatment regimen for peripheral neuropathy uses vibroacoustic stimulation to increase the production of adult stem cell to repair myelin, repair nerve cells, improve nerve fiber conduction, and provide better blood circulation in the treatment area. The treatment area includes the location where an affected peripheral nerve originates and along the length of the nerve.

The treatment regimen for peripheral neuropathy consists of number (typically 5) of HF passes followed by a number of LF passes (typically 2), followed by 5 one-second FF periods with modulation at 400 Hz at the end of application. The HF pass includes constant amplitude and frequency pulses that vary in frequency from pulse to pulse. The modulation frequency ranges from 1400 Hz to minimum 100 Hz per during the HF pass. The frequency applied during the LF pass varies from 4 Hz to 30 Hz. The application concludes with 5 one-second 5 periods modulated at 400 Hz. There is a rest period of at least 5 seconds between each HF pass.

The amplitude of HF modulated pulses is constant during each pulse and varies from pulse to pulse from 200 to 1000 microns. The amplitude of stimulation during the LF pass varies from 500 to 2000 microns. The total application time is from 18 to 20 minutes.

Parkinson's disease. Parkinson's disease (PD) belongs to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination.

The treatment regimen for Parkinson's disease will use vibroacoustic stimulation to increase adult stem cell production to improve function of dopamine-producing neurons, improve nerve cell conduction and increase blood circulation in the treatment area.

The treatment regimen includes two applications. The first application is applied along vertebral column staring from C1 vertebral body down to L1 vertebral body and 3 inches wide to the right and to the left from vertebral midline or direct on the vertebral column as shown in FIG. 7B. The first application includes a number passes of HF passes, a number of LF passes, and 5 one-second pulses with fixed frequency (FF) modulation of 400 Hz at the end of application. The sequence of HF and LF passes is as follows: 2 passes of HF followed by 1 pass of LF. After those four passes, there will be a 30 second rest period. Then 1 pass of HF followed by five one-second pulses with fixed frequency (FF) modulation of 400 Hz at the end of application. The duration per application should not exceed 15 minutes. The amplitude of micro-vibration varies from 200 microns to 1000 microns.

The second application consists of one or two HF passes applied to the head as shown in FIGS. 8B and 9B. The HF pass includes constant frequency pulses that vary in frequency from pulse to pulse. The modulation frequency starts at 1100 Hz and ends at 800 Hz or in reverse order from 800 Hz to 1100 Hz. The duration of each pulse can vary from 2 seconds to 3 seconds. The number of modulated pulses is from 30 to 60 pulses. The duration of the second application is from 1 to 3 minutes. The amplitude of HF modulated pulses varies from 50 to 200 microns.

Functional Constipation. Constipation is defined as having a bowel movement fewer than three times per week. Functional constipation means that the bowel is healthy but not working properly. Colonic inertia, delayed transit, and pelvic floor dysfunction are three types of functional constipation. Colonic inertia and delayed transit are caused by a decrease in muscle activity in the colon. These syndromes may affect the entire colon or may be confined to the lower, or sigmoid, colon. Pelvic floor dysfunctions are caused by a weakness of the muscles in the pelvis surrounding the anus and rectum. However, because this group of muscles is voluntarily controlled to some extent, biofeedback training is somewhat successful in retraining the muscles to function normally and improving the ability to have a bowel movement.

Functional constipation that stems from problems in the structure of the anus and rectum is known as anorectal dysfunction, or anismus. These abnormalities result in an inability to relax the rectal and anal muscles that allow stool to exit.

The treatment regimen #1 for colonic inertia uses vibroacoustic stimulation to stimulate autonomous and somatic nervous system to cause colonic muscle activation. Treatment regimen #2 for pelvic floor dysfunction uses vibroacoustic stimulation to stimulate somatic nervous system to cause pelvic muscle activation Treatment regimen #3 for anorectal dysfunction, or anismus uses vibroacoustic stimulation to stimulate autonomous and somatic nervous system to cause colonic muscle and pelvic muscle relaxation.

Treatment regiment #1 consists of 4 or 5 HF passes with modulated frequency starting from 1300 Hz and ending with frequency of 600 Hz or in reverse order from 600 Hz to 1300 Hz. The duration of each pulse can vary from 1 seconds to 2 seconds. The duration for application is between 12 and 15 minutes. The transducers are placed 8 inches above ramie pubis (pelvic bone in front) and 8 inches wide to the left from abdominal midline. Additional transducers can be placed in the area from the base of the crotch to the anus 2 inches wide to the right and 2 inches wide to the left from pelvic midline. This regimen can be applied to both areas simultaneously or with separate treatments.

Treatment regiment #2 consists of a number of HF passes of pulses modulated by High Frequency (HF) and Low Frequency (LF) without modulation, and ending application with 5 pulses of fixed frequency modulation of 400 Hz. The duration of each pulse during the HF pass is approximately 1 second. The sequence order of HF and LF passes is as follows: 1 LF pass starting at 3 Hz and ending at 100 Hz or in reverse order from 100 Hz to 3 Hz. The duration of LF passes is approximately 5 minutes. After the LF pass is finished there is a rest period of approximately 10 seconds followed by 3 HF passes. The modulation frequencies for HF pulses starts at 1500 Hz and end at 200 Hz. The duration of each HF pass is from 2.5 to 3 minutes with a rest period of about 10 seconds between passes. The HF passes are followed by another LF pass, followed by 5 one-second FF periods with modulation at 400 Hz at the end of application. The duration for application is between 24 and 26 minutes. The transducers placed in the area from the base of the crotch to the tale bone 2 inches wide to the right and 2 inches wide to the left from pelvic midline.

Treatment regiment #3 consists of number of HF passes, a number of LF passes, and ends application with 5 one-second FF periods with modulation at 400 Hz. The duration of each pulse during the HF pass is approximately 2 seconds. The sequence order of HF and LF is as follows: 3 HF passes with modulation frequencies ranging from 1500 Hz to 100 Hz or in reverse order from 100 Hz to 1500 Hz. The HF passes are followed by one LF pass in which the frequency ranges from 3 Hz to 100 Hz or in reverse order from 100 Hz to 3 Hz. The duration of the LF pass is approximately 3 to 5 minutes followed by 5 one-second FF periods with modulation at 400 Hz at the end of application. The amplitude of HF modulated pulses varies from 200 to 1000 microns. The amplitude of the stimulation applied during the LF pass varies from 500 to 2000 microns. The application time is from 20 to 22 minutes. The transducers placed in the area from the base of the crotch to the tale bone 2 inches wide to the right and 2 inches wide to the left from pelvic midline.

Urge incontinence (Over Active bladder). Urge incontinence is a sudden, intense urge to urinate, followed by an involuntary loss of urine. The bladder muscle contracts and may give a warning of only a few seconds to a minute to reach a toilet. With urge incontinence, there may also be a need to urinate often, sometimes several times a night. Some people with urge incontinence have a strong desire to urinate when they hear water running or after they drink only a small amount of liquid. Simply going from sitting to standing may even cause urine to leak. Urge incontinence may be caused by a urinary tract infection or by anything that irritates the bladder. It can also be caused by bowel problems or damage to the nervous system associated with multiple sclerosis, Parkinson's disease, Alzheimer's disease, stroke or injury. In urge incontinence, the bladder is said to be “overactive”—it's contracting even when your bladder isn't full. In fact, urge incontinence is often called an overactive bladder.

The treatment regimen for urge incontinence uses vibroacoustic stimulation to suppress autonomous nervous system to cause bladder muscle relaxation. The regimen consists of number (approximately 5) LF passes, followed by 1 HF, followed by 5 one-second FF periods with modulation at 400 Hz at the end of application. The LF passes start from 3 Hz and end with 200 Hz or in reverse order from 200 Hz to 3 Hz. The duration of each LF pass is around 3 minutes. The modulation frequencies for the HF pass starts from 1500 Hz and end at 200 Hz. The duration of HF pass is from 3 to 4 minutes follow by 5 one-second pulses with fix frequency modulation of 400 Hz at the end of application. The amplitude of HF modulated pulses varies from 100 to 500 microns. The amplitude of LF modulated pulses varies from 500 to 2000 microns. The duration of the application is from 18 to 22 minutes. The transducers are placed 6 inches above ramie pubis (pelvic bone in front) and 6 inches wide to the right and 6 inches wide to the left from abdominal midline. This treatment is also effective for anemia associates with a chronic condition, such as renal anemia.

Essential tremor. Essential tremor is an unintentional, somewhat rhythmic muscle movement involving to-and-fro movements (oscillations) of one or more parts of the body. Essential tremor (sometimes called benign essential tremor) is the most common of the more than 20 types of tremor. The treatment regimen for essential tremor uses vibroacoustic stimulation to stimulate motor nerve system. The regimen consists of one or two HF passes with modulated frequency starting from 1200 Hz and ending with frequency of 800 Hz or in reverse order from 800 Hz to 1200 Hz. The duration of each pulse can vary from 1.5 seconds to 2 seconds. The number of modulated pulses is from 30 to 60 pulses. The duration of the application is from 2 to 3 minutes. The amplitude of HF modulated pulses varies from 50 to 300 microns. Location is the same as for migraine headache as shown on FIGS. 8B and 9B.

In addition, micro-vibration treatment regimens have been developed for application to specific areas of the body.

Micro-vibration Treatment Regimen No. 1. The application area for Micro-vibration Treatment Regimen No. 1 includes the bone marrow and spinal cord located on or along vertebral column staring from C1 vertebral body down to L1 vertebral body and 3 inches wide to the right and to the left from vertebral midline as shown in FIG. 7B.

The therapeutic effects for Micro-vibration Treatment Regimen No. 1 include decreased mitosis time leading to increased stem cell multiplication in the application area; mobilization of stem cells and migration of stem cells into peripheral circulation in the application area; stem cells reaching peripheral circulation exhibiting less differentiation with more plasticity; and increased conductivity and enhanced signal to noise ratio in neural pathways in the spinal cord.

The application algorithm for Micro-vibration Treatment Regimen No. 1 includes one or more modulated multi-pulse application cycles referred to as a high frequency pass (HF pass). Each HF pass typically lasts from 1 to 5 minutes with an average of about 3 minutes per HF pass. The total minimum number of HF passes during an application can range from 1 to 12 HF passes with the lengths of the HF passes varying and having an average time of about 3 minutes per pass. The total duration of each application should be in the range of 3 minutes (for one HF pass) and up to about 60 minutes total. The average recommended duration of the application is from 15 to 60 minutes. Applications can be repeated with several hours between applications. Typical regimens include applications daily or several times per day for as long as the therapeutic effect is desired, typically an extended period of weeks, months or years.

The frequency range for Micro-vibration Treatment Regimen No. 1 is 1500 Hz to 600 Hz, which may decrease in 100 Hz pulse-to-pulse increments from 1500 HZ to 600 HZ during a HF pass, or it may increase from 600 Hz to 1500 Hz in 100 Hz pulse-to-pulse increments during a HF pass. The amplitude of the excitation ranges from 50 to 1000 microns and may change with frequency. For example, the amplitude may ramp from 100 microns at 1500 Hz to 1000 microns at 600 Hz, or the amplitude may ramp from 1000 microns at 600 Hz to 100 microns at 1500 Hz. The pulse width duration is typically from 0.1 to 5 seconds for each pulse, and the rest time between pulses is typically from 0.01 sec to 0.1 seconds.

Micro-vibration Treatment Regimen No. 2. The application area for Micro-vibration Treatment Regimen No. 2 includes the bone marrow and spinal cord located on or along vertebral column staring from C1 vertebral body down to L1 vertebral body and 3 inches wide to the right and to the left from vertebral midline as shown in FIG. 7B.

The therapeutic effects for Micro-vibration Treatment Regimen No. 2 include stem cells mobilization and forced to peripheral circulation and decreased conductivity of neural pathways in spinal cord depending on application duration.

The application algorithm for Micro-vibration Treatment Regimen No. 2 includes the application of pulses with or without modulation from 1.0 second to 0.08 seconds and from 0.08 seconds to 1.0 second. Each pass duration is from 1 to 5 minutes. The Low Frequencies (LF) pass consists of from 1 Hz to 120 Hz and from 120 Hz to 1 Hz. Total minimum number of LF passes 1 to 12, where is low frequencies smoothly changed from 1 Hz to 120 Hz or from 120 Hz to 1 Hz. The duration each LF pass no less than 1 minute and no more than 5 minutes. Application should not be less when 1 minutes and no longer than 60 minutes. Typical regimens include applications daily or several times per day for as long as the therapeutic effect is desired, typically an extended period of weeks, months or years.

The frequency range for Micro-vibration Treatment Regimen No. 2 is 1 Hz to 120 Hz or 120 Hz to 1 Hz non-modulated low frequency. The amplitude of the micro-vibration in microns range from about 10 up to 1000 Microns in the sweep from 1 Hz to 120 Hz or from 120 Hz to 1 Hz non-modulated. Pulse Width during Sweep range from 1 sec to 0.08 sec for sweep from 1 Hz to 120 Hz and from 0.08 sec to 1 sec for a sweep from 120 Hz to 1 Hz.

Micro-vibration Treatment Regimen No. 3. The application area for Micro-vibration Treatment Regimen No. 3 includes the bone marrow n and spinal cord located on or along vertebral column staring from C1 vertebral body down to L1 vertebral body and 3 inches wide to the right and to the left from vertebral midline as shown in FIG. 7B.

The therapeutic effects for Micro-vibration Treatment Regimen No. 3 include decreased mitosis time leading to increase stem cell multiplication in the application area; stem cell mobilization and migration of stem cells into peripheral circulation; stem cells entering peripheral circulation that are less differentiated with more plasticity; and increased conductivity and enhanced signal to noise ratio in neural pathways in spinal cord or decrease conductivity of neural pathways in spinal cord depending on preponderance of duration and quantity of HF and LF passes.

The application algorithm for Micro-vibration Treatment Regimen No. 3 consists of a mix of regimens described in Micro-vibration Treatment Regimen Nos. 1 and 2 in any order. Typical regimens include applications daily or several times per day for as long as the therapeutic effect is desired, typically an extended period of weeks, months or years.

The frequency range for Micro-vibration Treatment Regimen No. 3 is 1500 to 600 Hz or 600 to 1500 Hz Modulated (High Frequency) and 1 Hz to 120 Hz or 120 Hz to 1 Hz non-modulated. The amplitude of micro-vibration in microns range from 50 to 1000 microns change with frequency from 100 microns at 1500 Hz and 1000 microns at 600 Hz. Pulse width during the sweep include a combination of pulse duration from 0.1 to 5 seconds for each pulse at modulated sweep (HF) and 1 sec to 0.08 sec for sweep from 1 Hz to 120 Hz and from 0.08 sec to 1 sec for a sweep from 120 Hz to 1 Hz (LF). Rest time between modulated pulses during HF pass (sweep) width minimum is 0.01 sec and max 0.1 sec. Typical regimens include applications daily or several times per day for as long as the therapeutic effect is desired, typically an extended period of weeks, months or years.

Micro-vibration Treatment Regimen No. 4. The application area for Micro-vibration Treatment Regimen No. 4 includes the bone marrow and spinal cord located on or along vertebral column staring from C1 vertebral body down to L1 vertebral body and 3 inches wide to the right and to the left from vertebral midline as shown in FIG. 7B. The therapeutic effects for Micro-vibration Treatment Regimen No. 4 includes decreased mitosis time leading to increase stem cell multiplication. Simultaneously stem cell located in bone marrow of the spinal cord experience faster mobilization and migrate into peripheral circulation less differentiated with more plasticity without stimulating nerve pathways in the application area.

The application algorithm for Micro-vibration Treatment Regimen No. 4 includes pulse widths from 1 to 5 seconds with modulation frequency from 1200 Hz to 800 Hz in desired increments to fit pulse width, where pulse duration can be change from pulse to pulse. This is referred to as the High Frequency (HF) pass. The time between each pulse inside HF has a pause from 0.1 sec to 2 seconds. Each pass is from 2 to 4 minutes long. Total minimum number of passes 5 (average 3 to 4 minutes per pass). The duration of the entire application should not be less when 2 minutes (for one pass) and not longer than 120 minutes. Average recommended duration for application from 15 to 30 minutes. Typical regimens include applications daily or several times per day for as long as the therapeutic effect is desired, typically an extended period of weeks, months or years.

The frequency range for Micro-vibration Treatment Regimen No. 4 is 1200-800 modulated or 800 Hz to 1200 Hz (HF pass) and 2 to 120 Hz or 120 to 2 Hz (LF pass) non-modulated. Amplitude of micro-vibration in microns range from 50 to 1000 microns change with frequency from 100 microns at 1200 Hz and 1000 microns at 800 Hz and up to 2000 Microns in the sweep from 2 Hz to 120 Hz non-modulated. Pulse width during the pass is from 0.1 to 5 seconds for each pulse at modulated HF sweep and for LF sweep from 0.5 to 0.08 seconds. Rest time between modulated pulses during HF pass ranges from 0.1 to 2.0 seconds between pulses with modulation during HF sweep and no pause during LF sweep from 2 Hz to 120 Hz non-modulated. The Algorithm consists of mix of regimen described in Micro-vibration Treatment Regimen Nos. 1 and 2 in any order.

Micro-vibration Treatment Regimen No. 5. The application area for Micro-vibration Treatment Regimen No. 5 includes bone marrow located on the skull and brain as shown in FIGS. 8B and 9B.

The therapeutic effects for Micro-vibration Treatment Regimen No. 5 include vibroacoustic stimulation of stem cells located in bone marrow causing decreased mitosis time leading to increased stem cell multiplication. This regimen can also be used to stimulate adult neural stem cells to decrease the occurrence and severity of headaches.

The application algorithm for Micro-vibration Treatment Regimen No. 5 includes a HF pass with pulse width 1 to 5 seconds with modulation frequency from 1100 Hz to 600 Hz in desired increments to fit the pulse width, where the pulse duration can be changed from pulse to pulse. The time between each pulse inside HF has a pause from 0.1 sec to 2 seconds. Each HF pass from 0.5 to 4 minutes long for a minimum number of 1 or 2 passes. The duration of the entire application should not be less than 0.5 minutes (for one pass) and not longer than 10 minutes. Typical regimens include applications daily or several times per day for as long as the therapeutic effect is desired, typically an extended period of weeks, months or years.

The frequency range for Micro-vibration Treatment Regimen No. 5 is 1100-600 or 600 to 1100 Hz modulated. The amplitude of the micro-vibration in microns ranges from 10 to 200 microns and changes with frequency. The pulse width ranges from 0.1 to 3 seconds for each pulse at modulated HF pulses. Rest time between modulated pulses during HF pass ranges from 0.1 to 3.0 seconds.

Micro-vibration Treatment Regimen No. 6. The application area for Micro-vibration Treatment Regimen No. 6 includes bone marrow located in the skull and brain as shown in FIGS. 8B and 9B.

The therapeutic effects for Micro-vibration Treatment Regimen No. 6 include vibroacoustic stimulation of stem cells located in bone marrow resulting in decreased mitosis time leading to increased stem cell multiplication. This regimen can also be used to help dissolve a cerebral hematoma.

The application algorithm for Micro-vibration Treatment Regimen No. 6 includes a HF pass with pulse width ranging from 1 to 5 seconds with modulation frequency ranging from 1500 Hz to 200 Hz in desired increments to fit pulse width, where pulse duration can be changed from pulse to pulse. The rest time between each pulse during the HF pass ranges from 0.1 sec to 2 seconds. The duration of each pass ranges from 2 to 4 minutes with a minimum number of 4 passes 4 (average 2 to 4 minutes per pass). The duration of entire application should not be less than 6 minutes (for one pass) and not longer than 15 minutes per application. Typical regimens include applications daily or several times per day for as long as the therapeutic effect is desired, typically an extended period of weeks, months or years.

The frequency range for Micro-vibration Treatment Regimen No. 6 ranges from 1500 to 100 Hz or 100 to 1500 Hz modulated (HF pass). The amplitude of micro-vibration in microns ranges from 100 microns at frequency 1500 Hz and 300 Microns at frequency 100 Hz. The pulse width ranges from 0.1 to 2 seconds for each pulse. The rest time between modulated pulses ranges from 0.1 to 3.0 seconds between pulses.

Micro-vibration Treatment Regimen No. 7. The application area for Micro-vibration Treatment Regimen No. 7 includes bone marrow located along vertebral column staring from L1 vertebral body down to S5 vertebral body and 3 inches wide to the right and to the left from vertebral midline as shown in FIG. 10B.

The therapeutic effects for Micro-vibration Treatment Regimen No. 7 include decreased mitosis time leading to increased stem cell multiplication; simultaneously stem cells located in bone marrow experience increased mobilization and migration into peripheral circulation less differentiated with more plasticity; improvement of blood circulation in application area.

The application algorithm for Micro-vibration Treatment Regimen No. 7 includes a HF pass with pulse width ranging from 1 to 5 seconds with modulation frequency ranging from 1200 Hz to 100 Hz in desire increment to fit pulse width, where the pulse duration can change from pulse to pulse. The time between each pulse of the HF pass should not be less than 0.01 seconds. Each pass extends from 2 to 4 minutes with a minimum number of 5 passes (average 3 min per pass). The duration of the entire application should not be less than 3 minutes (for one pass) and not longer than 120 minutes. The average recommended duration for the application is 15 to 30 minutes. Typical regimens include applications daily or several times per day for as long as the therapeutic effect is desired, typically an extended period of weeks, months or years.

The frequency range for Micro-vibration Treatment Regimen No. 1 is 1200-100 Hz or 100-1200 Hz modulated. The amplitude of micro-vibration in microns ranges from 100 microns to 1000—at frequency 1200 Hz and up to 2000 Microns at frequency 100 Hz. The pulse width ranges from 0.1 to 7 seconds. The rest time between modulated pulses ranges from 0.1 to 1.0 seconds between pulses.

Micro-vibration Treatment Regimen No. 8. The application area for Micro-vibration Treatment Regimen No. 8 includes bone marrow located along vertebral column staring from L1 vertebral body down to S5 vertebral body and 3 inches wide to the right and to the left from vertebral midline as shown in FIG. 10B.

The therapeutic effects for Micro-vibration Treatment Regimen No. 8 include stem cells mobilization and migration into peripheral circulation. Pain is reduced in application area.

The application algorithm for Micro-vibration Treatment Regimen No. 8 includes LF pass ranging from 1 Hz changing up to 100 Hz. The amplitude of micro-vibration can vary from 100 microns to 2000 microns and may depend on the type of transducer used. The pulse duration ranges from 1.5 second to 0.1 second with rest time between pulses ranging from 0.01 to 0.1 seconds. Typical regimens include applications daily or several times per day for as long as the therapeutic effect is desired, typically an extended period of weeks, months or years.

The frequency range for Micro-vibration Treatment Regimen No. 8 is from 1 Hz to 100 Hz or 100 Hz to 1 Hz non-modulated (LF pass). The amplitude of micro-vibration in microns ranges from 100 to 2000 microns during LF sweep from 1 Hz to 100 Hz or 100 Hz to 1 Hz. Rest time between modulated pulses during HF passes (sweep) from 0.01 to 1 seconds. Rest time between passes of LF pulses without modulation from 0.1 to 1 seconds.

Micro-vibration Treatment Regimen No. 9. The application area for Micro-vibration Treatment Regimen No. 9 includes bone marrow located along vertebral column staring from L1 vertebral body down to S5 vertebral body and 3 inches wide to the right and to the left from vertebral midline as shown in FIG. 10B.

The therapeutic effects for Micro-vibration Treatment Regimen No. 9 include stem cells mobilization and migration into peripheral circulation and reduced pain in application area.

The application algorithm for Micro-vibration Treatment Regimen No. 9 includes a combination of HF and LF passes. The duration of the HF modulated pulses can range from 0.1 sec to 7 seconds with a total duration of the HF sweep ranging from 3 to 10 minutes. The duration of the HF sweep may be altered depending on the type of application and weight of the person receiving the treatment. Persons having lower weight typically receive treatments with shorter duration or lower amplitude of microvibration. The rest time between pulses ranges from 0.1 to 2 second. The rest time should be sufficient to allow reduced polarization during stimulated chemical reaction inside the scull and a release of stem cells into the peripheral circulation. Typical regimens include applications daily or several times per day for as long as the therapeutic effect is desired, typically an extended period of weeks, months or years.

The frequency range for Micro-vibration Treatment Regimen No. 9 ranges from 2000 to 100 modulated or 100 to 2000 Hz (HF pass) and 1 to 100 Hz or 1 to 100 Hz (LF pass) non-modulated. The amplitude of micro-vibration in microns ranges from 100 to 2000 microns during LF pass 1 to 100 Hz or 1 to 100 Hz non-modulated and 100 to 1000 on HF pass 1200 to 100 or 100 to 1200 Hz modulated. The pulse widths during the HF sweep ranges from 0.1 to 7 seconds and from 1 second to 0.01 second during the LF pass. The rest time between modulated pulses during HF pass ranges from 0.1 to 1 second between pulses during the HF pass and 0.1 to 0.2 sec for the LF pass.

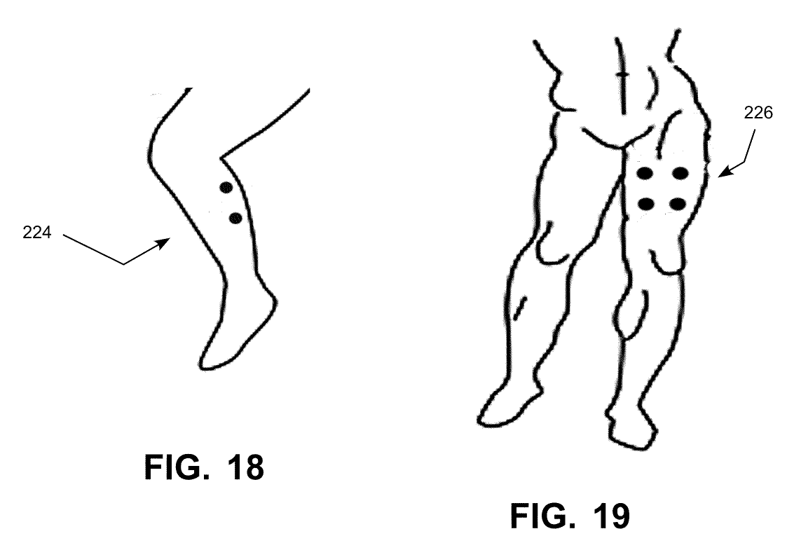

Micro-vibration treatments may also be used to aid in the recovery of injured muscles, tendons and ligaments in general and more particularly as occur in sports-related injuries. Regular micro-vibration treatments may also be used to improve blood flow to muscles as a training enhancement. As examples, FIG. 18 is a graphical representation of specific points 224 for applying micro-vibration treatments to stimulate the calf muscle and FIG. 19 is a graphical representation of specific points 226 for applying micro-vibration treatments to stimulate the thigh muscle. Of course, other target areas may also be treated, such as biceps, chest, abdominal muscles, and so forth. In general, micro-vibration treatments improve blood flow, encourage adult stem cell production, and encourage muscle growth and healing. The treatments can therefore be beneficial for a wide range of injury recovery, physical therapy, and athletic training programs. The treatment regimen typically includes multiple HF passes with modulation in the range from 400 to 1200 Hz, pulse amplitude in the range of 2000 to 50000 microns or higher if possible, pulse duration in the range of 1 to 2 seconds, HF pass duration in range of 1 to 3 minutes, total treatment 15 to 30 minutes, as least 4 hours between treatments, and treatments are typically applied at least once daily during the period of training or physical therapy.

Give the wide range of treatment areas and applications; the invention includes a wide range of devices for holding the micro-vibration transducers in place. For each type of treatment, one or more transducers are preferably held directly against the skin for the greatest transduction of acoustical pulse into the target tissue. In general, the transducers may be attached in any suitable way to any suitable applicator, such as a pillow, mattress pad, chair pad, wrap, strap or garment. The applicator may include pockets or other fasteners for removable holding the transducers to the application. Small pocket can be sewn into the applicators in the desired locations. Hook-and-loop (e.g., VELCRO®) fasteners have been found to be and effective and convenient way to removable hold the transducers in place, which allows the transducers to be moved around on the applications as desired. In particular, the exterior of a pillow, mattress pad, chair back pad, chair seat wrap, strap or garment can be covered with the relatively soft hook portion of the hook-and-loop fastener, while the relatively stiff hook portion is glued to the transducers. For difficult to reach locations, such the crotch area, neck, feet and so forth, the transducers may be attached to various locations on pillows having different shapes, which the user can sit on, lie against, lean against, or hold in place to position the transducers in the desired locations on the body.

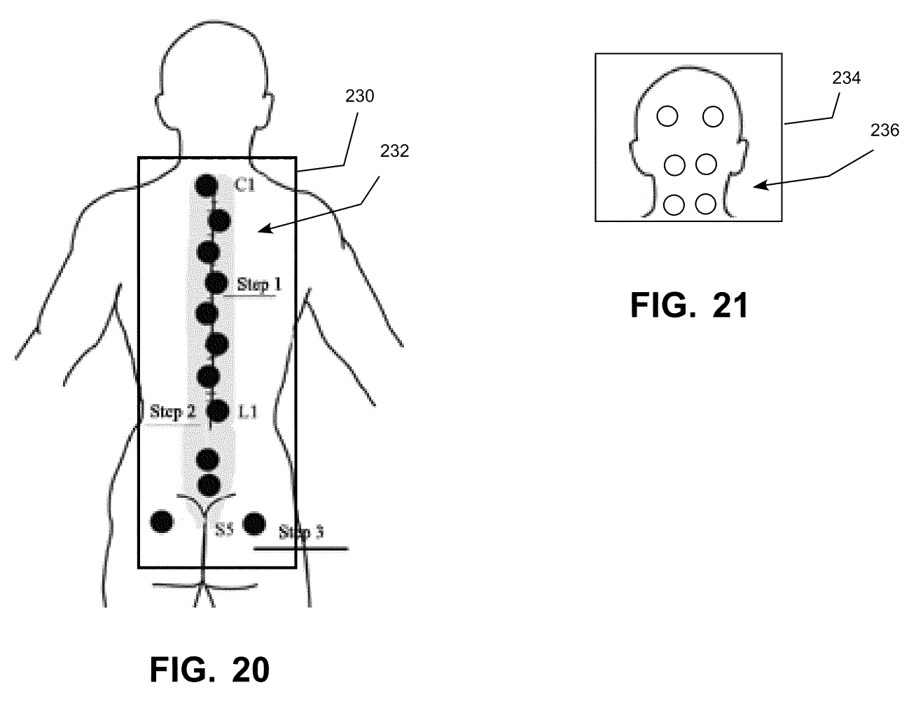

FIGS. 20-29 illustrate several examples of applicators for holding the acoustical transducers in place on desired locations of the body. FIG. 20 is a conceptual illustration of a pad 230 for holding micro-vibration transducers 232 in place for applying treatment to the back. The pad 230 may be laid on a bed or cot, for example, with the user lying on the pad as supported by the bed or cot. The pad 230 may have pockets for holding the transducers in desired locations. Alternatively, the pad may be covered with hook material allowing the transducers carrying hook material to be positioned in different locations on the pad. FIG. 21 is a conceptual illustration of a pillow or pillowcase 234 for holding the micro-vibration transducers 236 in place for applying treatment to the head and neck.

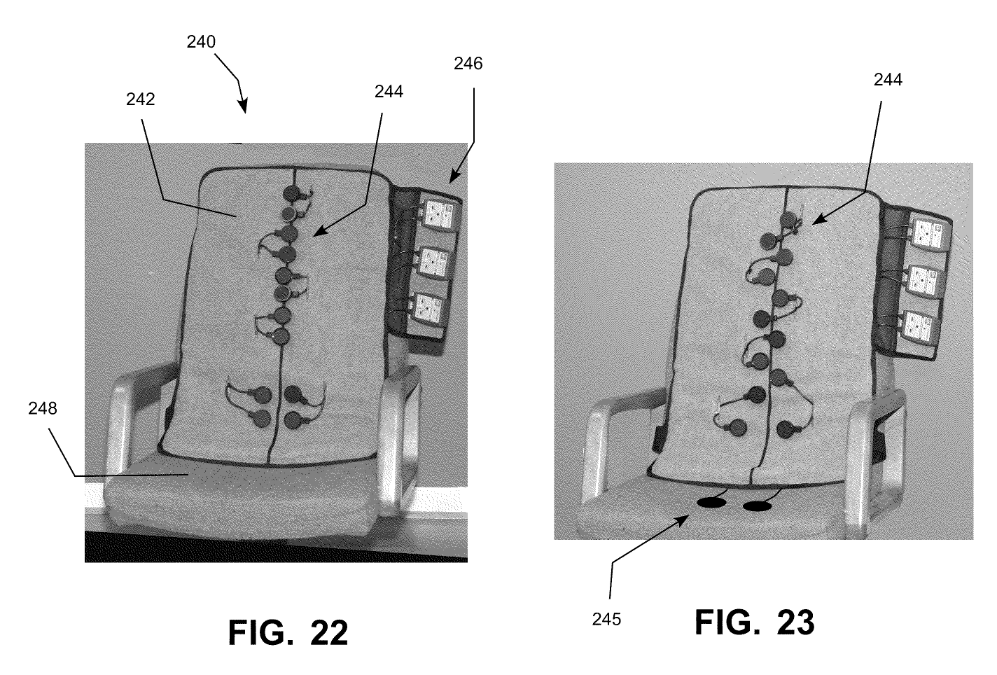

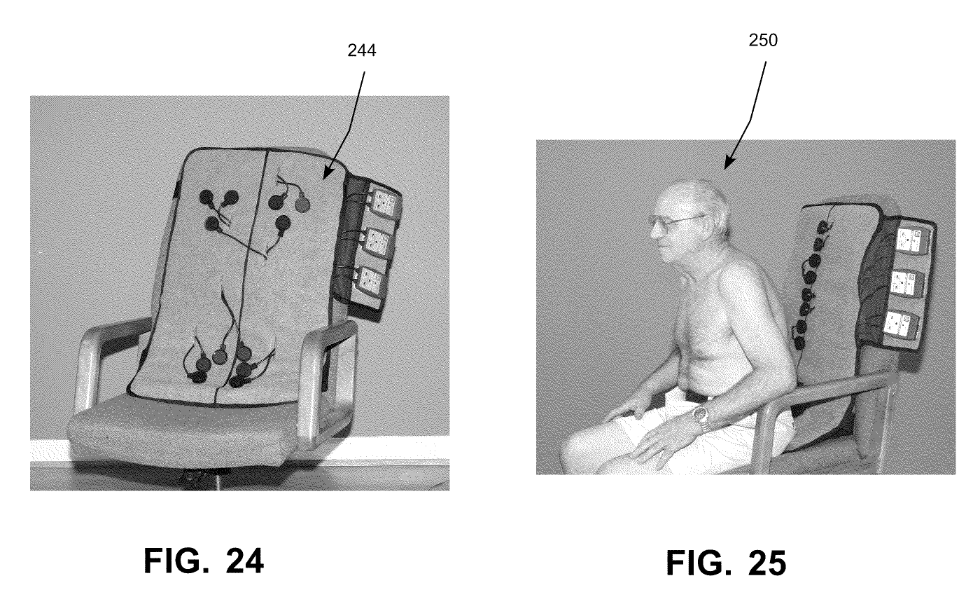

FIG. 22 is a conceptual illustration of a pad 240 attached to a chair holding micro-vibration transducers in place for applying treatment to the back. The pad includes a cover 242 of loop material for attaching the transducers 244 carrying hook material to be positioned in different locations on the pad. The wiring for the transducers preferably runs through and behind a cushion in the chair pad. This embodiment includes three controllers 246 for driving the transducers. As show in FIGS. 23 and 24, the transducers 244 may be moved into different locations on the pad. Transducers 245 may also be included on the chair seat 248 as desired. A neck support and headrest capable of supporting the transducers may also be added. FIG. 25 shows a user 250 moving into position on the chair pad.

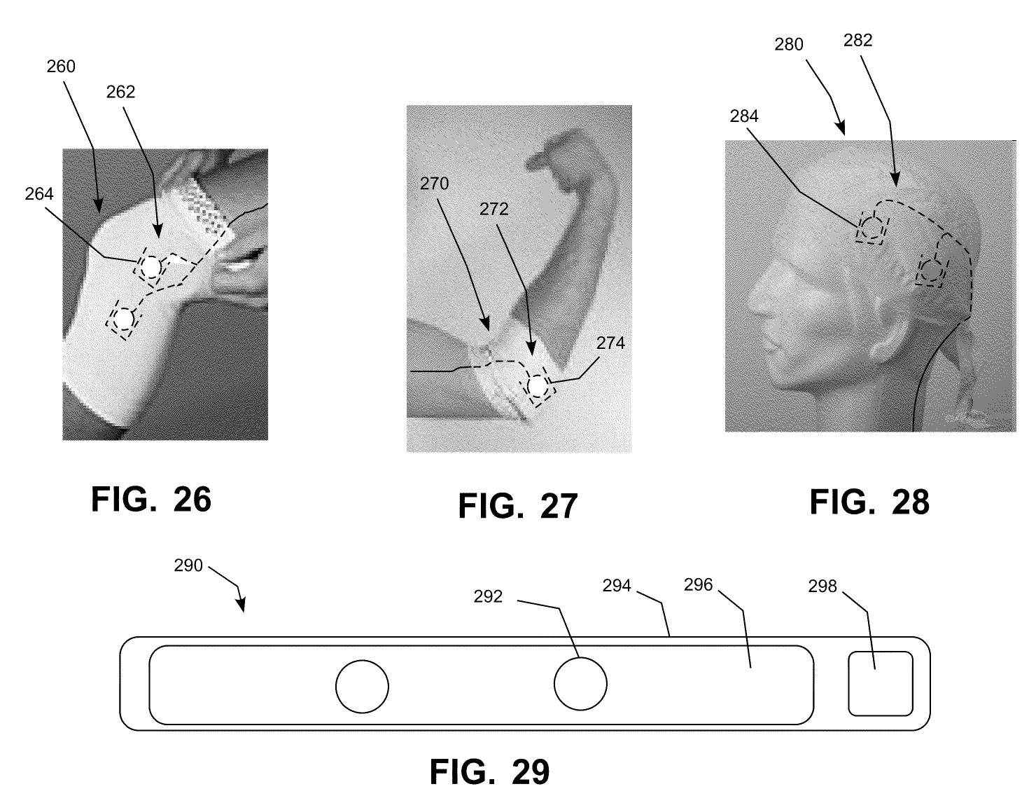

FIG. 26 is conceptual illustrations of a knee wrap 260 holding representative micro-vibration transducers 262 in place for applying treatment to the knee. In this embodiment, the transducers are held in place on the interior of the knee wrap by sewn pockets 264, although hook-and-loop fasteners may be used. For an elastic wrap of this type, the attachment devices may be omitted allowing the snug fir of the wrap itself to hold the transducers in place. The attachment devices may be desires as helpful aids in locating the transducers in the proper location. FIG. 27 shows a similar type of elbow wrap 270 holding a representative transducer 272 in place in a sewn pocket 274. FIG. 28 shows a similar type of head wrap 270 holding representative transducers 282 held in place in sewn pocket 284. For a more generally applicator, FIG. 29 shows is a conceptual illustration of a multi-purpose strap 290 holding representative transducers 292 in place. This particular strap includes a sturdy backing material 294 carrying a layer of hook material 296 and a piece of adhesive or hook material 298. The user may wrap or hold the strap in place over a desired treatment area, for example over the liver, neck, ankle or and injured area on the body. Gloves, socks, underwear and many other garments may be similarly fashioned as transducer applicators in accordance with the present invention.

It should be understood that the preceding regimens are illustrative of the types of treatments that have been found to be therapeutic, but that the specific parameters of the treatment may be varied within the scope of the invention as defined by the following claims. In view of the foregoing, it will be appreciated that present invention provides significant improvements for stimulating the growth of adult stem cells for a variety of therapeutic purposes.

The invention claimed is:

1. A method for applying a therapeutic treatment to target tissue areas of a living organism, comprising the steps of:
Placing one or more acoustical transducers on or near the external skin surface overlying or adjacent to the target tissue areas of the living organism;
Causing the acoustical transducers to produce a vibroacoustic signal profile comprising sequences of pulses defining a therapeutic regimen configured to impart a therapeutic effect to counteract a specific disease or condition affecting the target tissue areas;
Wherein the specific disease or condition is selected from the group consisting of multiple sclerosis, migraine headache, benign prostatic hyperplasia, spinal cord injury, peripheral neuropathy, Parkinson's disease, and essential tremor;
Wherein each pulse has a duration in the range from approximately one-half of a second to approximately ten seconds, each pulse has an amplitude in the range from approximately 20 microns to approximately 5,000 microns, and adjacent pulses are separated by rest periods in the range from approximately one-tenth of a second to approximately three seconds; and
Wherein the therapeutic regimen comprises one or more modulated frequency passes comprising pulses that are modulated with an oscillatory signal in the frequency range from approximately 3 Hz to approximately 1,200 Hz.

2. The method of claim 1, wherein:
The specific disease or condition comprises multiple sclerosis;
The target tissue areas include a vertebral column area and a head area;
The therapeutic regimen comprises a first application applied to the vertebral column area having a duration between approximately 10 minutes to approximately 60 minutes; and
The therapeutic regimen comprises a second application applied to the head area having duration between approximately 2 minutes to approximately 3 minutes.

3. The method of claim 2, wherein the first application comprises:
A plurality of modulated frequency passes, wherein each modulated frequency pass has duration from approximately 2 minutes to approximately 5 minutes and comprises a plurality of pulses, each pulse having a duration from approximately 2 seconds to approximately 5 seconds and comprising a modulation frequency from approximately 200 Hz to approximately 1100 Hz.

4. The method of claim 3, wherein the second application comprises at least one modulated frequency pass comprising a plurality of pulses, each pulse has a duration from approximately 1 second to approximately 2 seconds and comprises a modulation frequency from approximately 600 Hz to approximately 1100 Hz and each pulse has an amplitude of vibration in the range from approximately 20 microns to approximately 1000 microns.

5. The method of claim 4, further comprising a rest period from approximately 1 minute to approximately 60 minutes between the first application and the second application.

6. The method of claim 5, wherein the therapeutic effect comprises stimulation of growth of adult stem cells in the target tissue areas.

7. The method of claim 1, wherein:
The specific disease or condition comprises migraine headache;
The target tissue areas include head and neck area;
The therapeutic regimen comprises at least one modulated frequency pass having a duration from approximately 0.5 minutes to approximately 3 minutes and comprising a plurality of pulses, each pulse having a duration from approximately 0.5 seconds to approximately 2 seconds and comprising a modulation frequency from approximately 700 Hz to approximately 1100 Hz and each pulse has an amplitude of vibration in the range from approximately 200 microns to approximately 1000 microns.

8. The method of claim 1, wherein:
The specific disease or condition comprises benign prostatic hyperplasia;
The target tissue areas include a pelvic area;
The therapeutic regimen has duration of approximately 15 minutes to approximately 35 minutes and comprises a plurality of modulated high frequency passes and low frequency passes;
Wherein each low frequency pass has a duration of approximately 3 minutes to approximately 5 minutes during which the vibroacoustic signal profile varies in the range from approximately 3 Hz to approximately 100 Hz; and each pulse has an amplitude of vibration in the range from approximately 500 microns to approximately 5000 microns;
Wherein each high frequency pass has a duration from approximately 3 minutes to approximately 5 minutes and comprises a plurality of pulses, each pulse having a duration of approximately from 1 second to 2 seconds and comprising a modulation frequency from approximately 400 Hz to approximately 1000 Hz and each pulse has an amplitude of vibration in the range from approximately 1000 microns to approximately 5000 microns.

9. The method of claim 1, wherein:
The specific disease or condition comprises spinal cord injury;
The target tissue areas include a vertebral column area;
The therapeutic regimen has duration of approximately 10 minutes to approximately 30 minutes and comprises a plurality of modulated frequency passes;
Wherein each modulated frequency pass comprises a plurality of modulated pulses having amplitude of micro vibration from approximately 300 microns to approximately 5000; and
Wherein each modulated frequency pass has a duration from approximately 2 minutes to approximately 6 minutes and comprises a plurality of pulses, each pulse having a duration from approximately 1 second to approximately 6 seconds and comprising a modulation frequency from approximately 400 Hz to approximately 1200 Hz.

10. The method of claim 1, wherein:
the specific disease or condition comprises peripheral neuropathy;
The target tissue areas include a nerve affected by peripheral neuropathy;
The therapeutic regimen has a duration of approximately 15 minutes to approximately 20 minutes and comprises a plurality of modulated high frequency passes and low frequency passes;
Wherein each low frequency pass comprises a vibroacoustic signal profile in the range from approximately 4 Hz to approximately 40 Hz and amplitude of microvibrations from approximately 500 microns to approximately 4000 microns; and each pass having a duration from approximately 2 minutes to approximately 3 minutes, and
Wherein each high frequency pass comprising a plurality of pulses, wherein each pulse comprises a modulation frequency from approximately 800 Hz to approximately 1200 Hz and amplitude from approximately 500 microns to approximately 5000 microns, and each pulse having a duration from approximately 0.5 seconds to approximately 1 second; each pass having a duration from approximately 1 minute to approximately 2 minutes.

11. The method of claim 1, wherein:
The specific disease or condition comprises Parkinson's and essential tremor; disease;
The target tissue areas include a vertebral column area and a head area;
the therapeutic regimen comprises an application comprising a vibroacoustic signal profile in the range from approximately 200 microns to approximately 3000 microns applied to the vertebral column for a duration of not more than approximately 15 minutes to 30 minutes; and
Wherein each modulated frequency pass comprising a plurality of pulses, wherein each pulse comprises a modulation frequency from approximately 500 Hz to approximately 1000 Hz, and each pulse having a duration from approximately 2 seconds to approximately 7 seconds;
The therapeutic regimen comprises a head area application comprising a vibroacoustic signal profile having amplitude of micro vibrations in the range from approximately 100 microns to approximately 1000 microns applied to the head for a duration from approximately 1 minute to approximately 3 minutes, and each pass having a duration from approximately 1 minute to approximately 3 minutes.

12. The method of claim 11, wherein the therapeutic effect comprises stimulation of growth of adult stem cells in the target tissue areas.

13. A method for applying a therapeutic treatment to target tissue areas of a living organism, comprising the steps of:
Placing one or more acoustical transducers on or near the external skin surface overlying or adjacent to the target tissue areas of the living organism;
Causing the acoustical transducers to produce a vibroacoustic signal profile comprising sequences of pulses defining a therapeutic regimen configured to impart a therapeutic effect to counteract a specific disease or condition affecting the target tissue areas.
Wherein the specific disease or condition is selected from the group consisting of functional constipation, urge incontinence, anemia associated with chronic disease, and renal anemia;
Wherein each modulated pulse has a duration in the range from approximately 0.5 seconds to approximately 10 seconds, each pulse has an amplitude in the range from approximately 200 microns to approximately 5,000 microns, and adjacent modulated pulses are separated by rest periods in the range from approximately 0.1 seconds to approximately 3 seconds; and
Wherein the therapeutic regimen comprises one or more modulated and non modulated frequency passes comprising pulses that are modulated with an oscillatory signal in the frequency range from approximately 100 Hz to approximately 1,500 Hz and non modulated oscillatory signal in the range from approximately 1 HZ to approximately 100 Hz.

14. The method of claim 13, wherein:
The specific disease or condition comprises functional constipation characterized by colonic inertia;
The target tissue areas include an abdominal area and a pelvic area;
The therapeutic regimen has a duration of approximately 12 minutes to approximately 25 minutes and comprises a plurality of modulated frequency passes, each modulated frequency pass comprising a plurality of pulses, wherein each pulse has duration from approximately 4 seconds to approximately 10 seconds and comprises a modulation frequency from approximately 300 Hz to approximately 1500 Hz.

15. The method of claim 13, wherein:
The specific disease or condition comprises functional constipation characterized by pelvic floor dysfunction;
The target tissue areas include a crotch area;
The therapeutic regimen has duration of approximately 24 minutes to approximately 26 minutes and comprises a plurality of high frequency passes and a plurality of low frequency passes;
Each high frequency pass a duration from approximately 2.5 minutes to approximately 3 minutes and comprising a plurality of pulses, wherein each pulse comprises a modulation frequency from approximately 200 Hz to approximately 1500 Hz and duration from approximately 3 seconds to approximately 8 second and amplitude of approximately 2000 microns to 5000 microns; and
Each low frequency pass comprises a vibroacoustic signal profile in the range from approximately 1 Hz to approximately 200 Hz, amplitude of approximately 2000 microns to 5000 microns, and duration of at least approximately 5 minutes.

16. The method of claim 13, wherein:
The specific disease or condition comprises functional constipation characterized by ano-rectal dysfunction or anismus;
The target tissue areas include a crotch area;
The therapeutic regimen has duration from approximately 20 minutes to approximately 22 minutes and comprises a plurality of modulated frequency passes;
Each modulated frequency pass has duration of approximately 2 minutes to 3 minutes and comprises a plurality of modulated pulses, wherein each pulse comprises a modulation frequency from approximately 400 Hz to approximately 1500 Hz and amplitude from approximately 100 microns to approximately 2000 microns.

17. The method of claim 13 wherein:
The specific disease or condition comprises urinary urge incontinence;
The target tissue areas include a low abdominal and pelvic area;
The therapeutic regimen has duration of from approximately 18 minutes to approximately 22 minutes and comprises a plurality of high frequency passes and one or more low frequency passes;
Wherein each high frequency pass has a duration from approximately 3 minutes to approximately 4 minutes and comprises a plurality of pulses, wherein each pulse comprises a modulation frequency from approximately 200 Hz to approximately 1500 Hz and amplitude from approximately 100 microns to approximately 500 microns; and
Wherein each low non modulated frequency pass comprises a vibroacoustic signal profile in the range from approximately 3 Hz to approximately 200 Hz, duration from approximately 1 minute to approximately 3 minutes, and amplitude from approximately 200 microns to approximately 2000 microns.

18. A device for applying a therapeutic treatment to target tissue areas of a living organism, comprising:
One or more acoustical transducers configured to be located on or near the external skin surface overlying or adjacent to the target tissue areas of the living organism;
Means for causing the acoustical transducers to produce a vibroacoustic signal profile comprising sequences of pulses defining a therapeutic regimen configured to impart a therapeutic effect to counteract a specific disease or condition affecting the target tissue areas;
Wherein the specific disease or condition is selected from the group consisting of multiple sclerosis, migraine headache, benign prostatic hyperplasia, spinal cord injury, peripheral neuropathy, Parkinson's disease, essential tremor, functional constipation, urge incontinence, anemia associated with chronic disease, and renal anemia;
Wherein each pulse has a duration in the range from approximately 1 seconds to approximately 0.08 seconds, each pulse has an amplitude in the range from approximately 20 microns to approximately 5,000 microns, and adjacent modulated pulses are separated by rest periods in the range from approximately 0.1 seconds to approximately 3 seconds; and
Wherein the therapeutic regimen comprises one or more modulated frequency passes comprising pulses that are modulated with an oscillatory signal in the frequency range from approximately 200 Hz to approximately 1,500 Hz and non modulated oscillatory signal in the range from approximately 1 HZ to approximately 100 Hz.

19. The device of claim 18 wherein:
The specific disease or condition is anemia associated with chronic disease or renal anemia;
The target tissue areas include a vertebral column area and pelvic area;
The therapeutic regimen has duration of approximately 15 minutes to approximately 30 minutes and comprises a plurality of modulated frequency passes;
Wherein each modulated frequency pass comprises a plurality of non-modulated pulses having amplitude of micro vibration from approximately 300 microns to approximately 4000; and
Wherein each modulated frequency pass has a duration from approximately 2 to approximately 5 minutes and comprises a plurality of pulses, each modulated pulse having a duration from approximately 1.2 second to approximately 10 seconds and comprising a modulation frequency from approximately 400 Hz to approximately 1300 Hz.

20. The device of claim 18, wherein the therapeutic effect comprises stimulation of growth of adult stem cells in the target tissue areas.
